SEQUENCE LISTING (12 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 143 aa, Homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 3 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 4 - protein, 140 aa, Artificial Sequence
  Actimmune
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGR

SEQ ID NO: 5 - DNA, 498 bp, Artificial Sequence
  Expression cassette optimized for expression of interferon gamma in CHO cells
atgaagtacacaagctatatcctggcctttcagctgtgcatcgtgctgggctccctgggctgctattgccaggacccttacgtgaaggaggccgagaacctgaagaagtactttaacgccggccacagcgatgtggccgacaatggcacactgtttctgggcatcctgaagaattggaaggaggagagcgatcggaagatcatgcagtcccagatcgtgtccttctatttcaagctgtttaagaatttcaaggacgatcagtccatccagaagtccgtggagaccatcaaggaggacatgaacgtgaagtttttcaatagcaataagaagaagagagacgatttcgagaagctgaccaattactccgtgacagacctgaacgtgcagagaaaggccatccacgagctgatccaggtgatggccgagctgtcccccgccgccaagaccggcaagagaaagagaagccagatgctgttcagaggcagacgggccagccag

SEQ ID NO: 6 - DNA, 19 bp, Artificial Sequence
  primer
gatggctggcaactagaag

SEQ ID NO: 7 - DNA, 19 bp, Artificial Sequence
  primer
tgtacggtgggaggtctat

SEQ ID NO: 8 - DNA, 33 bp, Artificial Sequence
  primer
gttcaggtctgtcacggtgtaattggtcagctt

SEQ ID NO: 9 - DNA, 33 bp, Artificial Sequence
  primer
aagctgaccaattacaccgtgacagacctgaac

SEQ ID NO: 10 - DNA, 33 bp, Artificial Sequence
  primer
catgatcttccgatcggtctcgttcttccaatt

SEQ ID NO: 11 - DNA, 33 bp, Artificial Sequence
  primer
aattggaagaacgagaccgatcggaagatcatg

SEQ ID NO: 12 - DNA, 30 bp, Artificial Sequence
  primer
gagtctagattacagcatctggcttctctt

(19) United States
(12) Patent Application Publication
(10) Pub. No.: US 2003/0138403 A1
(43) Pub. Date: Jul. 24, 2003

(54) Interferon formulations

(75) Inventor: Joern Drustrup, Farum (DK)

Correspondence Address: MAXYGEN, INC., 515 GALVESTON DRIVE, RED WOOD CITY, CA, 94063, US

(73) Assignee: Maxygen ApS, Hoersholm (DK)

(21) Appl. No.: 10/186,962

(22) Filed: Jun. 28, 2002

Related U.S. Application Data
(60) Provisional application No. 60/302,140, filed on Jun. 29, 2001.
(60) Provisional application No. 60/316,170, filed on Aug. 30, 2001.
(60) Provisional application No. 60/357,945, filed on Feb. 19, 2002.

(51) Int. Cl. A61K 38/21

(52) U.S. Cl. 424/85.4; 514/58; 424/85.5; 424/85.6; 424/85.7

(57) ABSTRACT

The invention relates to interferon compositions, such as pharmaceutical interferon compositions and methods of their preparation. In particular it relates to stabilized compositions comprising an interferon molecule and a sulfoalkyl ether cyclodextrin derivative.

### CROSS-REFERENCE TO RELATED APPLICATIONS

This application claims priority from and benefit of U.S. Provisional Application No. 60/302,140 filed Jun. 29, 2001, U.S. Provisional Application No. 60/316,170 filed Aug. 30, 2001, and U.S. Provisional Application No. 60/357,945 filed Feb. 19, 2002, the disclosures of each which are incorporated herein by reference in their entirety for all purposes.

### COPYRIGHT NOTIFICATION

Pursuant to 37 C.F.R. §1.71 (e), Applicants note that a portion of this disclosure contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to interferon compositions, such as pharmaceutical interferon compositions and methods of their preparation. In particular it relates to stabilized compositions comprising an interferon molecule and a sulfoalkyl ether cyclodextrin derivative.

### BACKGROUND OF THE INVENTION

Interferons are important cytokines characterized by antiviral, antiproliferative, and immunomodulatory activities. These activities form a basis for the clinical benefits that have been observed in a number of diseases, including hepatitis, various cancers and multiple sclerosis. The interferons are divided into the type I and type II classes. Type I interferons includes interferons α (alpha), β (beta), τ (tau) and ω (omega), whereas interferon γ (gamma) is the only known member of the distinct type II class. Interferons are reviewed by Aggarwall and Gutterman, in Human Cytokines, Vol I, Blackwell Science, Inc. 1996.

Interferon beta and variants and conjugates thereof are described in WO 01/15736, and in PCT/DK02/00128, the contents of which are incorporated herein by reference. Interferon gamma is a cytokine produced by T-lymphocytes and natural killer cells and exists as a homodimer of two noncovalently bound polypeptide subunits. The mature form of each monomer comprises 143 amino acid residues (shown as SEQ ID NO:2) and the precursor form thereof, including the signal sequence, comprises 166 amino acid residues (shown as SEQ ID NO:3).

Each subunit has two potential N-glycosylation sites (Aggarwal et al., Human Cytokines, Blackwell Scientific Publications, 1992) at positions 25 and 97. Depending on the degree of glycosylation the molecular weight of interferon gamma in dimer form is 34-50 kDa (Farrar et al., Ann. Rev. Immunol, 1993, 11:571-611).

The primary sequence of wild-type human interferon was reported by Gray et al. (Nature 298:859-863, 1982), Taya et al. (EMBO J. 1:953-958, 1982), Devos et al. (Nucleic Acids Res. 10:2487-2501, 1982) and Rinderknecht et al. (J. Biol. Chem. 259:6790-6797, 1984), and in EP 77670, EP 89676 and EP 110044.

Interferon gamma variants and conjugates thereof are described in WO 01/36001, the content of which is incorporated herein by reference.

Experimental 3D structures of wild-type human interferon gamma determined by X-ray crystallography have been reported by Ealick et al. Science 252:698-702 (1991) who reported the C-alpha trace of an interferon gamma homodimer. Walter et al. Nature 376:230-235 (1995) disclosed the structure of an interferon gamma homodimer in complex with two molecules of a soluble form of the interferon gamma receptor. The coordinates of this structure, however, have never been made publicly available. Thiel et al. Structure 8:927-936 (2000) showed the structure of an interferon gamma homodimer in complex with two molecules of a soluble form of the interferon gamma receptor having a third molecule of the receptor in the structure not making interactions with the interferon gamma homodimer.

As a pharmaceutical compound recombinant human interferon gamma is used with a certain success, above all, against some viral infections and tumors. Recombiant human interferon gamma is usually applicable via parenteral, preferably via subcutaneous, injection.

One problem recognized in connection with formulation of interferons into pharmaceutical products is aggregation of the interferon molecules. This problem has been attempted solved by use of various stabilizers e.g. as described in WO 98/28007, WO 99/15193 and WO 01/24814. Commercially available interferon beta products have been stabilized by human serum albumin.

### BRIEF DESCRIPTION OF THE INVENTION

According to the invention novel interferon containing pharmaceutical compositions are provided, which compositions comprise a sulfoalkyl ether cyclodextrin derivative as a stabilizing agent.

Accordingly, in a first aspect the invention relates to a stabilized pharmaceutical composition comprising an interferon molecule and a sulfoalkyl ether cyclodextrin derivative.

The term “interferon molecule” is intended to indicate a polypeptide exhibiting interferon activity, e.g. as defined by Aggarwel and Gutterman, op cit.

The interferon molecule is typically selected from the group consisting of interferon alpha, interferon beta, interferon omega, interferon tau, interferon epsilon and interferon gamma.

In a further aspect the invention relates to a primary product container comprising an interferon molecule and a sulfoalkyl ether cyclodextrin derivative.

In a further aspect the invention relates to a method for increasing stability of an interferon molecule formulated into a pharmaceutical composition, said method comprising incorporating into said composition a sulfoalkyl ether cyclodextrin derivative and optionally a buffering agent.

In a further aspect the invention relates to a method of subjecting a mammal to interferon therapy, which method comprises administering an effective amount of a composition comprising an interferon molecule and a sulfoalkyl ether cyclodextrin derivative.

In a further aspect the invention relates to a method of treating a mammal with a disease, disorder, or condition for which interferon is a useful treatment, comprising administering to the mammal an effective amount of a composition comprising an interferon molecule and a sulfoalkyl ether cyclodextrin derivative.

In a further aspect the invention relates to a pharmaceutical composition comprising an interferon molecule and a sulfoalkyl ether cyclodextrin derivative.

In a further aspect the invention relates to use of a composition comprising an interferon molecule and a sulfoalkyl ether cyclodextrin derivative for the manufacture of a medicament for treatment of a disease, disorder, or condition for which interferon is a useful treatment or may potentially be a useful treatment.

When the interferon molecule is interferon alpha, or interferon beta, or a variant or conjugate thereof, this invention provides compositions and methods for treating a disease, disorder, or condition for which interferon alpha or interferon beta is a useful treatment or may potentially be a useful treatment, such as, for example: most types of viral infections, cancers or tumors or tumour angiogenesis, Crohn's disease, ulcerative colitis, Guillain-Barré syndrome, glioma, idiopathic pulmonary fibrosis, abnormal cell growth, or for immunomodulation in any suitable animal, preferably mammal, and in particular human. For example, the composition of the invention may be used in the treatment of osteosarcoma, basal cell carcinoma, ovarian carcinoma, cervical dysplasia, cervical carcinoma, laryngeal papillomatosis, mycosis fungoides, glioma, acute myeloid leukemia, multiple myeloma, Hodgkin's disease, melanoma, breast carcinoma, non-small cell lung cancer, malignant melanoma (adjuvant, late stage, as well as prophylactic), carcinoid tumour, B-cell lymphoma, T-cell lymphoma, follicular lymphoma, Kaposi's sarcoma, chronic myelogenous leukaemia, renal cell carcinoma, recurrent superfiecial bladder cancer, colorectal carcinoma, hairy cell leukaemia, and viral infections such as papilloma virus, viral hepatitis, herpes genitalis, herpes zoster, herpetic keratitis, herpes simplex, viral encephalitis, cytomegalovirus pneumonia, rhinovirus chronic persistent hepatitis, chronic active HCV (type I), chronic active HCV (type II) and chronic hepatitis B. In particular the molecule or composition of the invention may be used for the treatment of multiple sclerosis (MS), such as any of the generally recognized four types of MS (benign, relapsing remitting MS (RRMS), primary progressive MS (PPMS) and secondary progressive MS (SPMS)) and for monosymptomatic MS), cancer or tumours, hepatitis, e.g. hepatitis B and hepatitis C, or a herpes infection (the latter treatment optionally being combined with a treatment with IL-10).

When the interferon molecule is interferon gamma or a variant or conjugate thereof, the composition of the invention may be used for treatment of a disease, disorder, or condition for which interferon gamma is a useful treatment or may potentially be a useful treatment, such as, for example, any of the medical indications described in WO 01/36001, in particular interstitial pulmonary diseases, most particularly idiopathic pulmonary fibrosis. Interferon gamma has been suggested for treatment of interstitial lung diseases (IPF) for which purpose interferon gamma can be used in combination with prednisolone. In addition to IPF, granulomatous diseases, certain mycobacterial infections, kidney cancer, osteopetrosis, scleroderma, hepatitis B, hepatitis C, septic shock, and rheumatoid arthritis may be treated with interferon gamma.

In further aspects the invention relates to a kit comprising a composition according to the invention.

### DETAILED DESCRIPTION OF THE INVENTION

Unless otherwise defined below or elsewhere in the specification, all technical and scientific terms used herein have the same meaning as commonly understood by those of ordinary skill in the art to which the invention belongs.

The term “stabilized” is intended to mean that the composition has increased storage stability as compared to a composition which does not comprise the sulfoalkyl ether cyclodextrin derivative. For instance, the increased storage stability is observed in a liquid formulation stored as such or stored in a frozen state and thawed prior to use, in a dried form, e.g. lyophilized, spray-dried or air-dried form, for later reconstitution into a liquid form prior to use, in a solid form, e.g. intented for pulmonary or nasal delivery, and/or in any other form, e.g. made for a special drug delivery system (such as microspheres or the like). The increased storage stability is typically measured in terms of increased bioactivity as compared to the reference composition when subjected to the same storage conditions. The increased storage stability is intended to comprise physical and/or chemical stability.

The term “bioactivity” is intended to mean an in vitro and/or in vivo bioactivity as determined by any suitable assay. For instance, bioactivity can be measured in terms of antiviral activity, antiproliferative activity, immunomodulatory activity, receptor binding/activation activity, etc. according to methods known in the art relevant for the interferon molecule of interest.

It has been found that sulfoalkyl ether cyclodextrin derivatives have a profound stabilizing activity on interferon molecules that form aggregates during storage. Therefore, the present invention finds particular use for stabilization of such interferon molecules. Also, interferon molecules which for other reasons loose activity during storage, e.g. as a consequence of chemical or other physical degradation during storage, may be stabilized according to the present invention.

The term “aggregate formation” is intended to mean a physical interaction between the interferon molecules that results in formation of oligomers. Aggregate formation is undesirable since in most cases this leads to reduced or even lost bioactivity and/or increased immunogenicity. When the composition of the invention is a liquid composition aggregates may remain soluble or be in the form of large visible aggregates that precipitate out of solution. When the composition is in dry form aggregates may have been formed during preparation thereof resulting in an inferior formulation. Such aggregate formation may be measured by visual inspection, or be measured in any suitable spectrophotometric device.

The Interferon Molecule

The present invention is generally applicable to all types of interferon including interferons isolated from natural sources (e.g. human leukocytes or fibroblasts), recombinantly produced naturally occurring or variant interferons, as well as chemically synthesized interferons. For instance, the interferon molecule may be, but is not limited to, a type I interferon or a type II interferon, e.g. selected from the group consisting of interferon alpha, interferon beta, interferon omega, interferon tau, interferon epsilon and interferon gamma.

The interferon molecule may have the amino acid sequence of that found in nature (wildtype interferon) or may be a variant of such wildtype interferon.

More specifically, the interferon molecule may be a variant of a wildtype interferon comprising one or more amino acid modifications, i.e. deletions, insertions or substitutions, and exhibiting interferon activity. Such modifications can be made in a site-specific manner (e.g. by use of site-directed mutagenesis) or in a random or semi-random manner, e.g. by use of random or localized random mutagenesis, e.g. as defined in WO 01/04287 or by use of directed evolution technology, e.g. as described by Stemmer, Bio/Technology 13:549-553 (1995), U.S. Pat. No. 5,605,793, U.S. Pat. No. 5,830,721, U.S. Pat. No. 5,811,238, etc. Normally, the variant comprises at most 15 amino acid modifications, e.g. 1, 2, 3, 4, 5, 6, 7, 8, 9, 10, 11, 12, 13, 14 or 15 modifications. Most preferably, the interferon molecule is a human interferon (having the amino acid sequence of a naturally occurring human interferon) or a variant thereof.

The term “conjugate” (or interchangeably “conjugated polypeptide”) is intended to indicate a heterogeneous (in the sense of composite or chimeric) molecule formed by the covalent attachment of one or more interferon polypeptide(s) to one or more non-polypeptide moieties. The term covalent attachment means that the interferon polypeptide and the non-polypeptide moiety are either directly covalently joined to one another, or else are indirectly covalently joined to one another through an intervening moiety or moieties, such as a bridge, spacer, or linkage moiety or moieties. Preferably, a conjugated interferon polypeptide is soluble at relevant concentrations and conditions, i.e. soluble in physiological fluids such as blood. Examples of conjugated interferon polypeptides include glycosylated and/or PEGylated interferon polypeptides. The term “non-conjugated polypeptide” may be used about the polypeptide part of the conjugated interferon polypeptide.

The term “one difference” or “differs from” as used in connection with specific modifications is intended to allow for additional differences being present apart from the specified amino acid difference. Thus, in addition to the introduction of the specific modifications disclosed herein the interferon polypeptides may, if desired, comprise other modifications. These may, for example, include addition of one or more extra residues at the N-terminus, e.g. addition of a Met residue at the N-terminus and/as truncation of one or more C-terminal residues as well as “conservative acid substitutions”, i.e. substitutions performed within groups of amino acids with similar characteristics, e.g. small amino acids, acidic amino acids, polar amino acids, basic amino acids, hydrophobic amino acids and aromatic amino acids. Examples of conservative substitutions may, in particular, be selected from the groups listed in the table below.

| | | | | |
|---|---|---|---|---|
| 1 | Alanine (A) | Glycine (G) | Serine (S) | Threonine (T) |
| 2 | Aspartic | Glutamic | | |
| | acid (D) | acid (E) | | |
| 3 | Asparagine (N) | Glutamine (Q) | | |
| 4 | Arginine (R) | Histidine (H) | Lysine (K) | |
| 5 | Isoleucine (I) | Leucine (L) | Methionine (M) | Valine (V) |
| 6 | Phenylalanine | Tyrosine (Y) | Tryptophan (W) | |
| | (F) | | | |

The term “random mutagenesis” refers to a mutagenic process that is random with respect to the site of mutation within the subject nucleic acid, and that is random with respect to the mutations introduced, e.g., chemical mutagenesis, uv or y irradiation, passage through repair deficient cells, etc. The term “localized mutagenesis” is used to indicate that the mutagenic process occurs preferentially in a predetermined portion or subsequence of the subject nucleic acid. In the context of the present invention, “site directed mutagenesis” refers to an alteration at a predetermined nucleotide position or positions, normally with the aim of altering one or more amino acid residues of the encoded amino acid sequence. The site-directed mutagenesis is normally designed on the basis of an analysis of a primary or tertiary (e.g. model) structure of the polypeptide to be modified.

The term “attachment group” is intended to indicate an amino acid residue group capable of coupling to the relevant non-polypeptide moiety such as a polymer molecule or a sugar moiety. Useful attachment groups and their matching non-polypeptide moieties are apparent from the table below.

| | | | Conjugation | |
|---|---|---|---|---|
| Attachment | | Examples of non- | method/ | |
| group | Amino acid | polypeptide moiety | Activated PEG | Reference |
| —NH₂ | N-terminal, Lys | Polymer, e.g. PEG | mPEG-SPA | Shearwater Inc. |
| | | | Tresylated | Delgado et al, |
| | | | mPEG | critical reviews |
| | | | | in Therapeutic |
| | | | | Drug Carrier |
| | | | | Systems |
| | | | | 9(3,4):249-304 |
| | | | | (1992) |
| —COOH | C-term, Asp, Glu | Polymer, e.g. PEG | mPEG-Hz | Shearwater Inc |
| | | Sugar moiety | In vitro coupling | |
| —SH | Cys | Polymer, e.g. PEG, | PEG- | Shearwater Inc |
| | | | vinylsulphone | Delgado et al, |
| | | | PEG-maleimide | critical reviews |
| | | Sugar moiety | In vitro coupling | in Therapeutic |
| | | | | Drug Carrier |
| | | | | Systems |
| | | | | 9(3,4):249-304 |
| | | | | (1992) |
| —OH | Ser, Thr, OH—, | Sugar moiety | In vivo O-linked | |
| | Lys | | glycosylation | |
| —CONH₂ | Asn as part of an | Sugar moiety | In vivo | |
| | N-glycosylation | | glycosylation | |
| | site | | | |
| Aromatic | Phe, Tyr, Trp | Sugar moiety | In vitro coupling | |
| residue | | | | |
| —CONH₂ | Gln | Sugar moiety | In vitro coupling | Yan and Wold, |
| | | | | Biochemistry, |
| | | | | 1984, Jul 31; |
| | | | | 23(16): 3759-65 |
| Aldehyde | Oxidized | Polymer, e.g. PEG, | PEGylation | Andresz et al., |
| Ketone | carbohydrate | PEG-hydrazide | | 1978, Makromol. |
| | | | | Chem. 179:301; |
| | | | | WO 92/16555, |
| | | | | WO 00/23114 |
| Guanidino | Arg | Sugar moiety | In vitro coupling | Lundblad and |
| | | | | Noyes, Chimical |
| | | | | Reagents for |
| | | | | Protein |
| | | | | Modification, |
| | | | | CRC Press Inc. |
| | | | | Boca Raton, FI |
| Imidazole | His | Sugar moiety | In vitro coupling | As for guanidine |
| ring | | | | |

For in vivo N-glycosylation, the term “attachment group” is used in an unconventional way to indicate the amino acid residues constituting an N-glycosylation site (with the sequence N-X′-S/T/C-X″, wherein X′ is any amino acid residue except proline, X″ any amino acid residue that may or may not be identical to X′ and that preferably is different from proline, N is asparagine and S/T/C is either serine, threonine or cysteine, preferably serine or threonine, and most preferably threonine). Although the asparagine residue of the N-glycosylation site is the one to which the sugar moiety is attached during glycosylation, such attachment cannot be achieved unless the other amino acid residues of the N-glycosylation site is present. Accordingly, when the non-polypeptide moiety is a sugar moiety and the conjugation is to be achieved by N-glycosylation, the term “amino acid residue comprising an attachment group for the non-polypeptide moiety” as used in connection with alterations of the amino acid sequence of the parent polypeptide is to be understood as one, two or all of the amino acid residues constituting an N-glycosylation site is/are to be altered in such a manner that either a functional N-glycosylation site is introduced into the amino acid sequence or removed from said sequence.

Obviously, removal and/or introduction of amino acid residues comprising an attachment group for a non-polypeptide moiety is primarily of interest when the interferon molecule is in the form of a conjugate having one or more attached non-polypeptide moieties. By removing and/or introducing amino acid residues comprising an attachment group for a non-polypeptide moiety it is possible to optimize the number and distribution of non-polypeptide moieties conjugated to the interferon molecule (e.g. to ensure an optimal distribution of non-polypeptide moieties on the surface of the interferon molecule and thereby, e.g., effectively shield epitopes and other surface parts of the polypeptide without significantly impairing the function thereof) as explained in further detail in WO 01/15736, and in PCT/DKO2/00128. For instance, by introduction of attachment groups, the interferon polypeptide is boosted or otherwise altered in the content of the specific amino acid residues to which the relevant non-polypeptide moiety binds, whereby a more efficient, specific and/or extensive conjugation is achieved. By removal of one or more attachment groups it is possible to avoid conjugation to the non-polypeptide moiety in parts of the polypeptide in which such conjugation is disadvantageous, e.g. to an amino acid residue located at or near a functional site of the polypeptide (since conjugation at such a site may result in inactivation or reduced interferon activity of the resulting conjugate due to impaired receptor recognition). Further, it may be advantageous to remove an attachment group located closely to another attachment group in order to avoid heterogeneous conjugation to such groups.

It will be understood that the amino acid residue comprising an attachment group for a non-polypeptide moiety, either it be removed or introduced, is selected on the basis of the nature of the non-polypeptide moiety and, in most instances, on the basis of the conjugation method to be used. For instance, when the non-polypeptide moiety is a polymer molecule, such as a polyethylene glycol or polyalkylene oxide derived molecule, amino acid residues capable of functioning as an attachment group may be selected from the group consisting of lysine, cysteine, aspartic acid, glutamic acid and arginine. When the non-polypeptide moiety is a sugar moiety the attachment group is an in vivo glycosylation site, preferably an N-glycosylation site.

The amino acid residue to be introduced or removed is normally located in a surface exposed position of the interferon molecule, preferably in a position that is occupied by an amino acid residue which has more than 25% of its side chain exposed to the solvent, preferably more than 50% of its side chain exposed to the solvent. Furthermore, it may be relevant to remove an attachment group occupying a position in the interferon molecule, which is located in a receptor binding site. Also, it may be relevant to introduce an attachment group into a position located in or at an epitope of the interferon molecule. Such positions can be identified on the basis of an analysis of a 3D structure of the interferon molecule or by any other suitable method, e.g. as described in WO 01/15736 wherein interferon beta is used as an example.

Substitutions that lead to introduction of an additional N-glycosylation site at positions exposed at the surface of the interferon beta molecule and occupied by amino acid residues having more than 25% of the side chain exposed to the surface include: S2N+N4S/T, L6S/T, L5N+G7S/T, F8N+Q10S/T, L9N+R11S/T, R11N, R11N+S13T, S12N+N14S/T, F15N+C17S/T, Q16N+Q18S/T, Q18N+L20S/T, K19N+L21S/T, W22N+L24S/T, Q23N+H25S/T, G26N+L28S/T, R27N+E29S/T, L28S+Y30S/T, Y30N+L32S/T, L32N+D34S/T, K33N+R35S/T, R35N+N37S/T, M36N+F38S/T, D39S/T, D39N+P41S/T, E42N+I44S/T, Q43N+K45S/T, K45N+L47S/T, Q46N+Q48S/T, L47N+Q49T/S, Q48N+F50S/T, Q49N+Q51S/T, Q51N+E53S/T, K52N+D54S/T, L57N+I59S/T, Q64N+I66S/T, A68N+F70S/T, R71N+D73S/T, Q72N, Q72N+S74T, D73N, D73N+S75T, S75N+T77S, S75N, S76N+G78S/T, E81N+I83S/T, T82N+V84S/T, E85N+L87S/T, L88S/T, A89N+V91S/T, Y92S/T, Y92N+Q94S/T, H93N+I95S/T, L98S/T, H97N+K99S/T, K99N+V 101 S/T, T100N+L102S/T, E103N+K105S/T, E104N+L106S/T, K105N+E107S/T, E107N+E109S/T, K108N+D110S/T, E109N+F111S/T, D110N+T112S, D110N, F111N+R113S/T, R113N+K115S/T, G114N+L116S/T, K115N+M117S/T, L116N, L116N+S118T, S119N+H212S/T, L120N+L122S/T, H121N+K123S/T, K123N+Y125S/T, R124N+Y126S/T, G127N+1129S/T, R128N+L130S/T, L130N+Y132S/T, H131N+L133S/T, K134N+K136S/T, A135N+E137S/T, K136N+Y138S/T, E137N, Y138N+H140S/T, H140N+A142S/T, V148N+1150S/T, R152N+F154S/T, Y155N+1157S/T, L160S/T, R159N+T161S, R159N, G162N+L164S/T, and Y163N+R165S/T, the substitutions being indicated relative to the amino acid sequence of wildtype human interferon beta shown in SEQ ID NO:1.

Substitutions that lead to introduction of an additional N-glycosylation site at positions exposed at the surface of the interferon beta molecule having more than 50% of the side chain exposed to the surface include: L6S/T, L5N+G7S/T, F8N+Q10S/T, L9N+R11S/T, S12N+N14S/T, F15N+C17S/T, Q16N+Q18S/T, K19N+L21S/T, W22N+L24S/T, Q23N+H25S/T, G26N+L28S/T, R27N+E29S/T, Y30N+L32S/T, K33N+R35S/T, R35N+N37S/T, M36N+F38S/T, D39S/T, D39N+P41S/T, E42N+I44S/T, Q46N+Q48S/T, Q48N+F50S/T, Q49N+Q51S/T, Q51N+E53S/T, K52N+D54S/T, L57N+I59S/T, R71N+D73S/T, D73N, D73N+S75T, S75N+T77S, S75N, S76N+G78S/T, E81N+183S/T, T82N+V84S/T, E85N+L87S/T, A89N+V91S/T, Y92S/T, Y92N+Q94S/T, H93N+195S/T, T100N+L102S/T, E103N+K105S/T, E104N+L106S/T, E107N+E109S/T, K108N+D110S/T, D110N+T112S, D110N, F111N+R113S/T, R113N+K115S/T, L116N, L116N+S118T, K123N+Y125S/T, R124N+Y126S/T, G127N+1129S/T, H131N+L133S/T, K134N+K136S/T, A135N+E137S/T, E137N, V148N+1150S/T, and Y155N+I157S/T, the substitutions being indicated relative to the amino acid sequence of wildtype human interferon beta shown in SEQ ID NO:1.

Among the substitutions mentioned in the above lists, those are preferred that have the N residue introduced among the 141 N-terminal amino acid residues, in particular among the 116 N-terminal amino acid residues.

Substitutions that lead to introduction of an N-glycosylation site by only one amino acid substitution include: L6S/T, R11N, D39S/T, Q72N, D73N, S75N, L88S/T, Y92S/T, L98S/T, D110N, L116N, E137N, R159N and L160S/T, the substitutions being indicated relative to the amino acid sequence of wildtype human interferon beta shown in SEQ ID NO:1. Among these, a substitution is preferred that is selected from the group consisting of L6S/T, R11N, D39S/T, Q72N, D73N, S75N, L88S/T, Y92S/T, L98S/T, D110N and L116N, more preferably from the group consisting of L6S/T, D39S/T, D73N, S75N, L88S/T, D110N, L116N and E137N; and most preferably selected from the group consisting of L6S/T, D39S/T, D73N, S75N, L88S/T, D110N and L116N.

Preferably, the introduced amino acid residue comprising an attachment group for a non-polypeptide moiety creates a new N-glycosylation site or a new PEGylation site.

Furthermore, when the interferon molecule comprises a glycosylation site the utilization of such site may be optimised. This can be achieved by modification of an amino acid residue located close to said glycosylation site, the modification being of a type resulting in an increasing glycosylation. Normally, the in vivo glycosylation site is an N-glycosylation site, but it can also be an O-glycosylation site.

In the present context the term “increased glycosylation” is intended to indicate increased levels of attached carbohydrate molecules, normally obtained as a consequence of increased (or better) utilization of glycosylation site(s). The increased glycosylation may be determined by any suitable method known in the art for analyzing attached carbohydrate structures.

In the present context the term “increased degree of in vivo N-glycosylation” or “increased degree of N-glycosylation” is intended to indicate increased levels of attached carbohydrate molecules, normally obtained as a consequence of increased (or better) utilization of N-glycosylation site(s). In case of interferon gamma, it is well-known (Hooker et al., 1998, J. Interferon and Cytokine Res. 18, 287-295 and Sarenva et al., 1995, Biochem J., 308, 9-14) that when wild-type human interferon gamma is expressed in CHO cells only about 50% of the interferon gamma molecules utilizes both glycosylation sites, about 40% utilizes one glycosylation site (1N), and about 10% is not glycosylated (₀N). The increased degree of in vivo N-glycosylation may be determined by any suitable method known in the art, e.g. by SDS-PAGE.

The term “exhibiting interferon gamma activity” is intended to indicate that the interferon gamma polypeptide has one or more of the functions of native human interferon gamma or recombinant human interferon gamma, including the capability to bind to an interferon gamma receptor and cause transduction of the signal transduced upon human interferon gamma-binding of its receptor as determined in vitro or in vivo (i.e. in vitro or in vivo bioactivity). The interferon gamma receptor has been described by Aguet et al. (Cell 55:273-280, 1988) and Calderon et al. (Proc. Natl. Acad. Sci. USA 85:4837-4841, 1988). A suitable assay for testing interferon gamma activity is the assay entitled “Primary Assay” disclosed herein.

An “interferon gamma polypeptide” (also referred to herein as an “interferon gamma molecule” or an “interferon gamma”) is a polypeptide exhibiting interferon gamma activity, and is used herein about the interferon gamma polypeptide in monomer or dimeric form, as appropriate. For instance, when specific substitutions are indicated these are normally indicated relative to the interferon gamma polypeptide monomer. It will be understood that the term “interferon gamma polypeptide” also encompasses C-terminally truncated and variant forms of the wild-type interferon gamma molecule. Specific examples of such variants include variants with modifications such as S99T, E38N+S40T as well as C-terminally truncated forms thereof. More examples of suitable modifications are given below.

Normally, the variant forms of the interferon gamma polypeptide differs in 1-15 amino acid residues (such as in 1, 2, 3, 4, 5, 6, 7, 8, 9, 10, 11, 12, 13, 14 or 15 amino acid residues), e.g. in 1-10 amino acid residues, in 1-5 amino acid residues or in 1-3 amino acid residues compared to human interferon gamma (or where a truncated form thereof is desired: compared to the corresponding truncated human interferon gamma).

As indicated above, it is known that 1-15 amino acid residues may be deleted from the C-terminus without abolishing interferon gamma activity of the molecule. Thus, the term “interferon gamma polypeptide” also encompasses interferon gamma polypeptides (having the human wild-type sequence or a variant thereof), wherein 1-15 the interferon gamma polypeptide is C-terminally truncated with 1-15 amino acid residues. One specific example include an interferon gamma polypeptide, which has been C-terminally truncated with 3 amino acid residues.

The term “human interferon gamma” is intended to mean the mature form of wild-type human interferon gamma having the amino sequence shown in SEQ ID NO:2.

The term “recombinant human interferon gamma” is intended to cover the mature form of wild-type human interferon gamma having the amino acid sequence shown in SEQ ID NO:2 which has been produced by recombinant means.

When used herein the term “Actimmune®)” refers to the 140 amino acid form of interferon gamma (disclosed in SEQ ID NO:4) achieved by fermentation of a genetically engineered E. coli bacterium. Further information of Actimmune® is available at www.actimmune.com.

An amino acid residue “located close to” a glycosylation site is usually located in position −4, −3, −2, −1, +1, +2, +3 or +4 relative to the amino acid residue of the glycosylation site to which the carbohydrate is attached. Such positions are selected from position −1, +1, or +3, in particular in position +1 or +3. Thus, the amino acid residue located close to an N-glycosylation site (having the sequence N-X′-S/T/C-X″) may be located in position −4, −3, −2, −1 relative to the N-residue, at position X′ or X″ (in which case the amino acid residue to be introduced is preferably different from proline), or at position +1 relative to the X″ residue. The amino acid modification is normally a substitution, the substitution being made with any other amino acid residue that gives rise to an increased glycosylation of the interferon molecule as compared to that of the unmodified molecule. Such other amino acid residue may be determined by trial and error type of experiments (i.e. by substitution of the amino acid residue of the relevant position to any other amino acid residue, and determination of the resulting glycosylation of the resulting variant).

When position +2 relative to the N-residue is modified it will be understood that only a limited number of modifications are possible since in order to maintain/introduce an in vivo N-glycosylation site, the amino acid residue in said position must be either Ser, Thr or Cys. In a particular preferred embodiment, the modification of the amino acid residue in position +2 relative to the in vivo N-glycosylation site is a substitution where the amino acid residue in question is replaced with a Thr residue. If, on the other hand, said amino acid residue is already a Thr residue it is normally not preferred or necessary to perform any substitutions in that position. When X′ is modified, X′ should not be Pro and preferably not Trp, Asp, Glu and Leu. If X′ is modified, the amino acid residue to be introduced is preferably selected form the group consisting of Phe, Asn, Gln, Tyr, Val, Ala, Met, Ile, Lys, Gly, Arg, Thr, His, Cys and Ser, more preferably Ala, Met, Ile, Lys, Gly, Arg, Thr, His, Cys and Ser, in particular Ala or Ser. When position +3 relative to the N-residue is modified, the amino acid residue to be introduced is preferably selected from the group consisting of His, Asp, Ala, Met, Asn, Thr, Arg, Ser and Cys, more preferably Thr, Arg, Ser and Cys. Such modifications are particular relevant if the X′ residue is a Ser residue.

Also, a free cysteine of the interferon molecule (i.e. a cysteine residue which does not form part of a cysteine bridge) may be removed, e.g. by substitution with another amino acid residue such as a neutral amino acid residue such as Gly, Val, Ala, Leu, Ile, Tyr, Phe, His, Trp, Ser, Thr or Met, preferably Ser or Thr, e.g. as described in U.S. Pat. No. 4,959,314 or EP 192 811.

The interferon molecule can be derivatized by a non-polypeptide moiety, e.g. a polymer molecule such as polyethylene glycol, or a sugar moiety (when the interferon molecule is glycosylated).

The term “non-polypeptide moiety” is intended to indicate a molecule that is capable of conjugating to an attachment group of the polypeptide of the invention. Preferred examples of such molecule include polymer molecules, sugar moieties, lipophilic compounds, or organic derivatizing agents. When used in the context of a conjugate of the invention it will be understood that the non-polypeptide moiety is linked to the polypeptide part of the conjugate through an attachment group of the polypeptide.

The term “polymer molecule” is defined as a molecule formed by covalent linkage of two or more monomers, wherein none of the monomers is an amino acid residue, except where the polymer is human albumin or another abundant plasma protein. The term “polymer” may be used interchangeably with the term “polymer molecule”. The term is intended to cover carbohydrate molecules attached by in vitro glycosylation, i.e. a synthetic glycosylation performed in vitro normally involving covalently linking a carbohydrate molecule to an attachment group of the polypeptide, optionally using a cross-linking agent. Carbohydrate molecules attached by in vivo glycosylation, such as N- or O-glycosylation (as further described below)) are referred to herein as “a sugar moiety”. Except where the number of non-polypeptide moieties, such as polymer molecule(s) or sugar moieties in the conjugate is expressly indicated every reference to “a non-polypeptide moiety” contained in a conjugate or otherwise used in the present invention shall be a reference to one or more non-polypeptide moieties, such as polymer molecule(s) or sugar moieties, in the conjugate.

When the interferon molecule comprises an introduced amino acid residue comprising an attachment group for a non-polypeptide moiety, the non-polypeptide moiety is preferably attached to such amino acid residue.

In a first aspect the invention relates to a stabilized composition comprising an interferon molecule and a sulfoalkyl ether cyclodextrin derivative.

In a second aspect the invention relates to a stabilized pharmaceutical composition comprising an interferon molecule and a sulfoalkyl ether cyclodextrin derivative.

In one embodiment the sulfoalkyl ether cyclodextrin derivative is present in a concentration from 1 mg/ml to 150 mg/ml, such as from 5 mg/ml to 100 mg/ml.

In one embodiment the interferon molecule comprises at least one introduced and/or at least one removed amino acid residue comprising an attachment group for a non-polypeptide moiety. In a further embodiment the interferon molecule comprises at least one introduced and at least one removed amino acid residue comprising an attachment group for a non-polypeptide moiety. In a further embodiment the interferon molecule comprises at least one introduced amino acid residue comprising an attachment group for a non-polypeptide moiety. In a further embodiment the interferon molecule comprises at least one removed amino acid residue comprising an attachment group for a non-polypeptide moiety.

In a further embodiment the non-polypeptide moiety comprises a polymer molecule, such as polyethylene glycol, or a sugar moiety. In a specific embodiment the polymer molecule comprises a polyethylene glycol. In another specific embodiment the non-polypeptide moiety comprises a sugar moiety, in particular the sugar moiety obtained from expression of the interferon molecule in a mammalian cell, preferably a CHO cell.

In a further embodiment the interferon molecule comprises at least one introduced glycosylation site, and at least one sugar moiety attached to an introduced glycosylation site. In a further embodiment the interferon molecule comprises at least one introduced glycosylation site, and at least one sugar moiety attached to an introduced glycosylation site, and a polymer molecule, such as polyethylene glycol. In a particular preferred embodiment the interferon molecule comprises 3 sugar moieties each sugar moiety being attached to an N-glycosylation site.

In a preferred embodiment, the interferon molecule is glycosylated and/or PEGylated. In a further embodiment, the interferon molecule is glycosylated and PEGylated. In a further embodiment, the interferon molecule is glycosylated. In a further embodiment, the interferon molecule is PEGylated. When the interferon molecule is glycosylated it is preferably N-glycosylated. When the interferon molecule is glycosylated it usually comprises 1-5 sugar moieties, such as 1-3 sugar moieties. In a further embodiment, the interferon molecule is N-glycosylated, and comprises 1-5 sugar moieties, such as 1-3 sugar moieties. When the interferon molecule is PEGylated it usually comprises 1-5 polyethylene glycol (PEG) molecules. In a further embodiment the interferon molecule comprises 1-5 PEG molecules, such as 1, 2 or 3 PEG molecules. In a further embodiment each PEG molecule has a molecular weight of about 5 kDa (kilo Dalton) to 100 kDa. In a further embodiment each PEG molecule has a molecular weight of about 10 kDa to 40 kDa. In a further embodiment each PEG molecule has a molecular weight of about 12 kDa. In a further embodiment each PEG molecule has a molecular weight of about 20 kDa. Preferably the interferon molecule comprises 1-3 PEG molecules each having a molecular weight of about 12 kDa, or 1 PEG molecule having a molecular weight of about 20 kDa. Suitable PEG molecules are available from Shearwater Polymers, Inc. and Enzon, Inc. and may be selected from SS-PEG, NPC-PEG, aldehyd-PEG, mPEG-SPA, mPEG-SCM, mPEG-BTC, SC-PEG, tresylated mPEG (U.S. Pat. No. 5,880,255), or oxycarbonyl-oxy-N-dicarboxyimide-PEG (U.S. Pat. No. 5,122,614).

In one preferred embodiment the interferon molecule is an interferon beta molecule (also referred to herein as an “interferon beta polypeptide” or an “interferon beta”), e.g. wildtype human interferon beta or a variant thereof, optionally conjugated to a non-polypeptide moiety.

For instance, the interferon beta molecule can be a variant of wildtype human interferon beta, wherein the cysteine residue in position 17 has been deleted or substituted with another amino acid residue, e.g. a neutral amino acid residue as mentioned above. For instance, the interferon beta molecule comprises the C17S mutation, the substitution being indicated relative to the amino acid sequence of wildtype human interferon beta shown in SEQ ID NO:1.

In a preferred embodiment the interferon beta molecule is any of those described in WO 01/15736. In another preferred embodiment the interferon beta molecule is any of those described in PCT/DK02/00128. Preferred variants include a variant with at least one introduced glycosylation site, e.g. in position S2, Q49, Q51, or F111; and a variant with at is least one removed PEGylation site, e.g. in position K19, K33, K45 or K123.

In a further embodiment the interferon beta molecule is a conjugate comprising at least one first non-polypeptide moiety covalently attached to an interferon β polypeptide, the amino acid sequence of which differs from that of wild-type human interferon β in at least one introduced and at least one removed amino acid residue comprising an attachment group for said first non-polypeptide moiety.

In a further embodiment the interferon beta molecule is a conjugate comprising at least one first non-polypeptide moiety conjugated to at least one lysine residue of an interferon β polypeptide, the amino acid sequence of which differs from that of wild-type human interferon β in at least one introduced and/or at least one removed lysine residue.

In a further embodiment the interferon beta molecule is a conjugate comprising at least one first non-polypeptide moiety conjugated to at least one cysteine residue of an interferon β polypeptide, the amino acid sequence of which differs from at least one introduce cysteine residue into a position that in wild-type human interferon β is occupied by a surface exposed amino acid residue.

In a further embodiment the interferon beta molecule is a conjugate comprising at least one first non-polypeptide moiety having an acid group as an attachment group, which moiety is conjugated to at least one aspartic acid or glutamic acid residue of an interferon β polypeptide, the amino acid sequence of which differs from that of wild-type human interferon β in at least one introduced and/or at least one removed aspartic acid or glutamic acid residue.

In a further embodiment the first non-polypeptide moiety comprises a polymer molecule, such as polyethylene glycol, or a sugar moiety.

In a further embodiment the interferon beta molecule is a conjugate comprising at least one polymer molecule and at least one sugar moiety covalently attached to an interferon β polypeptide, the amino acid sequence of which differs from that of wild-type human interferon β in at least one introduced and/or at least one removed amino acid residue comprising an attachment group for the polymer molecule, and at least one introduced and/or at least one removed amino acid residue comprising an attachment group for the sugar moiety, provided that when the attachment group for the polymer molecule is a cysteine residue, and the sugar moiety is an N-linked sugar moiety, a cysteine residue is not inserted in such a manner that an N-glycosylation site is destroyed.

In a further embodiment the interferon beta molecule is a conjugate comprising an interferon β polypeptide, the amino acid sequence of which differs from that of wild-type human interferon β in at least one introduced glycosylation site, the conjugate further comprising at least one un-PEGylated sugar moiety attached to an introduced glycosylation site.

In a further embodiment the interferon beta molecule is a conjugate comprising an interferon β polypeptide, the amino acid sequence of which differs from that of wild-type human interferon β in at least one introduced glycosylation site, the conjugate further comprising at least one sugar moiety attached to an introduced glycosylation site.

Thus, in a particular aspect the invention relates to a stabilized composition comprising:

a) a conjugate comprising an interferon β polypeptide, the amino acid sequence of which differs from that of wild-type human interferon β in at least one introduced glycosylation site, the conjugate further comprising at least one sugar moiety attached to an introduced glycosylation site, and

b) a sulfoalkyl ether cyclodextrin derivative.

In a further embodiment the interferon beta molecule is a conjugate comprising an interferon β polypeptide, the amino acid sequence of which differs from that of wild-type human interferon β in that a glycosylation site has been introduced or removed by way of introduction or removal of amino acid residue(s) constituting a part of a glycosylation site in a position that in wildtype human interferon β is occupied by a surface exposed amino acid residue.

In a further embodiment the interferon beta molecule is a conjugate comprising an interferon β polypeptide, the amino acid sequence of which differs from that of wild-type human interferon β in that a glycosylation site has been introduced by way of introduction of amino acid residue(s) constituting a part of a glycosylation site in a position that in wildtype human interferon β is occupied by a surface exposed amino acid residue.

In a still further embodiment the interferon beta molecule is a conjugate comprising a sugar moiety covalently attached to an interferon β polypeptide, the amino acid sequence of which differs from that of wild-type human interferon β in at least one removed glycosylation site.

In a still further embodiment the interferon beta molecule is a glycosylated variant of a parent interferon β polypeptide comprising at least one in vivo glycosylation site, wherein an amino acid residue of said parent polypeptide located close to said glycosylation site has been modified to obtain a variant polypeptide having increased glycosylation as compared to the glycosylation of the parent interferon β polypeptide.

In a further embodiment the interferon beta molecule comprises amino acid substitutions selected from the group consisting of K19R+K45R+K123R; K19Q+K45R+K123R; K19R+K45Q+K123R; K19R+K45R+K123Q; K19Q+K45Q+K123R; K19R+K45Q+K123Q; K19Q+K45R+K123Q; K19Q+K45Q+K123Q; K45R+K123R; K45Q+K123R; K45Q+K123Q; K45R+K123Q; K19R+K123R; K19Q+K123R; K19R+K123Q; K19Q+K123Q; K19R+K45R; K19Q+K45R; K19R+K45Q; K19Q+K45Q; K52R+K134R; K99R+K136R; K33R+K105R+K136R; K52R+K108R+K134R; K99R+K115R+K136R; K19R+K33R+K45R+K123R; K19R+K45R+K52R+K123R; K19R+K33R+K45R+K52R+K123R; K19R+K45R+K52R+K99R+K123R; K19R+K45R+Q49N+Q51T+F111N+R113T+K123R; K19R+K45R+Q49N+Q51T+F111N+R113T; K19R+K45R+Q49N+Q51T+K123R; S2N+N4T/S; L9N+R11T/S; R11N; S12N+N14T/S; F15N+C17S/T; Q16N+Q18T/S; K19N+L21T/S; Q23N+H25T/S; G26N+L28T/S; R27N+E29T/S; L28N+Y30T/S; D39T/S; K45N+L47T/S; Q46N+Q48T/S; Q48N+F50T/S; Q49N+Q51T/S; Q51N+E53T/S; R71N+D73T/S; Q72N; D73N; S75N; S76N+G78T/S; L88T/S; Y92T/S; N93N+I95T/S; L98T/S; E103N+K105T/S; E104N+L106T/S; E107N+E109T/S; K108N+D110T/S; D110N; F111N+R113T/S; L116N; S2N+N4T; L9N+R11T; 49N+Q51T; F111N+R111N+113T; R71N+D73T; 49N+Q51T; F111N+R113T; R71N+D73T; Q49N+Q51T+F111N+R113T; Q49N+Q51T+R71N+D73T+F111N+R113T; S2N+N4T+F111N+R113T; S2N+N4T+Q49N+Q51T; S2N+N4T+Q49N+Q51T+F111N+R113T; S2N+N4T+L9N+R11T+Q49N+Q51T; S2N+N4T+L9N+R11T+F111N+R13T; S2N+N4T+L9N+R11 T+Q49N+Q51T+F111N+R113T; L9N+R11T+Q49N+Q51T; L9N+R11T+Q49N+Q51T+F111N+R113T; L9N+R11T+F111N+R113T; R27K^{+R}159K; R27K+K45R+R159K; R27K+Q49K+E85K+A89K; R27K+K45R+Q49K+E85K+A89K; R27K+D39K+Q49K+E85K+A89K; R27K+D39K+K45R+Q49K+E85K+A89K; N4K+R27K+D39K+Q49K+E85K+A89K; N4K+R27K+D39K+K45R+Q49K+E85K+A89K; R27K+K123R+R159K; R27K+K45R+K123R+R159K; R27K+Q49K+E85K+A89K+K123R; R27K+K45R+Q49K+E85K+A89K+K123R; R27K+D39K+Q49K+E85K+A89K+K123R; R27K+D39K+K45R+Q49K+E85K+A89K+K123R; N4K+R27K+D39K+Q49K+E85K+A89K+K123R; N4K+R27K+D39K+K45R+Q49K+E85K+A89K+K123R; K19R+K45R+F111K+K123R; K19R+K45R+Q49K+F111K+K123R; K19R+K45R+Q49K+K123R; K19R+K45R+F111K; K19R+K45R+Q49K+F111K; K19R+Q49K+K123R; K19R+Q49K+F111K+K123R; K45Q+F111K+K123Q; K45R+Q49K+K123R; and K45R+Q49K+F111K+K123R; the substitutions being indicated relative to the amino acid sequence of wildtype human interferon beta shown in SEQ ID NO: 1. Each of the specified variants is considered an embodiment of the interferon beta variant.

Specific interferon beta variants of interest for the present invention comprises amino acid substitutions selected from the group consisting of

Q49N+Q51T;

Q49N+Q51T+F111N+R113T;

F111N+R113T;

C17S+Q49N+Q51T+L98P+F111N+R113T;

S2N+N4T+C17S+Q51N+E53T;

S2N+N4T+C17S+Q51N+E53T+F111N+R113T;

C17S+Q49N+Q51T+F111N+R113T;

C17S+Q49N+Q51T+D110F+F111N+R113T;

C17S+Q48F+Q49N+Q51T+D110F+F111N+R113T;

C17S+Q48Y+Q49N+Q51T+D110Y+F111N+R113T;

K19R+K45R+K123R;

K19R+K45R+Q49N+Q51T+F111N+R113T+K123R;

C17S+K19R+K45R+Q49N+Q51T+F111N+R113T+K123R;

C17S+K19R+K45R+Q49N+Q51T+F111N+R113T+K123R;

C17S+K19R+Q49N+Q51T+F111N+R113T+K123R;

C17S+K19R+K45R+Q49N+Q51T+D110F+F111N+R113T+K123R;

C17S+K19R+Q49N+Q51T+D110F+F111N+R113T+K123R;

S2N+N4T+C17S+K19R+K45R+Q51N+E53T+K123R;

C17S+K19R+K45R+Q48F+Q49N+Q51T+D110F+F111N+R113T+K123R;

S2N+N4T+C17S+K19R+K45R+Q51N+E53T+D110F+F111N+R113T+K123R;

C17S+K19R+K33R+K45R+Q49N+Q51T+D110F+F111N+R113T;

C17S+K19R+K33R+K45R+Q49N+Q51T+D110F+F111N+R113T+K123R;

C17S+K19R+K33R+K45R+Q49N+Q51T+F111N+R113T; and

C17S+K19R+K33R+K45R+Q49N+Q51T+F111N+R113T+K123R; the substitutions being indicated relative to the amino acid sequence of wildtype human interferon beta shown in SEQ ID NO:1. Each of the specified variants is considered an embodiment of the interferon beta variant. For instance, one embodiment is C17S+Q49N+Q51T+D110F+F111N+R113T; and another embodiment is C17S+K19R+K33R+K45R+Q49N+Q51T+D110F+F111N+R113T. Particularly preferred embodiments of the above specified variants are such wherein the indicated substitutions are the only substitutions relative to the wildtype human interferon beta shown in SEQ ID NO:1.

These variants may be in the form of conjugates, which further comprise one or more non-polypeptide moieties. For instance the variant is glycosylated and/or PEGylated. When the interferon beta molecule is glycosylated it is preferably N-glycosylated. When the interferon beta molecule is glycosylated it usually comprises 1-5 sugar moieties, such as 1-3 sugar moieties. In a further embodiment, the interferon beta molecule is N-glycosylated, and comprises 1-5 sugar moieties, such as 1-3 sugar moieties.

When the interferon beta molecule comprises at least one introduced glycosylation site it is preferred that the molecule further comprises at least one sugar moiety attached to the introduced glycosylation site. In particular the interferon beta molecule comprises 2-5 sugar moieties, such as 2-3 sugar moieties. In a further embodiment, the interferon beta molecule is N-glycosylated, and comprises 2-5 sugar moieties, such as 2-3 sugar moieties. In a particular preferred embodiment the interferon beta molecule comprises 3 sugar moieties each sugar moiety being attached to an N-glycosylation site.

When the interferon beta molecule is PEGylated it usually comprises 1-5 polyethylene glycol (PEG) molecules. In a further embodiment the interferon beta molecule comprises 1-5 PEG molecules, such as 1, 2 or 3 PEG molecules. In a further embodiment each PEG molecule has a molecular weight of about 5 kDa (kilo Dalton) to 100 kDa. In a further embodiment each PEG molecule has a molecular weight of about 10 kDa to 40 kDa. In a further embodiment each PEG molecule has a molecular weight of about 12 kDa. In a further embodiment each PEG molecule has a molecular weight of about 20 kDa. Preferably the interferon beta molecule comprises 1-3 PEG molecules each having a molecular weight of about 12 kDa, or 1 PEG molecule having a molecular weight of about 20 kDa. More preferably the interferon beta molecule comprises at least one sugar moiety attached to an introduced glycosylation site and 1 PEG molecule having a molecular weight of about 20 kDa. Most preferably the interferon beta molecule comprises 3 sugar moieties attached to 3 N-glycosylation sites and 1 PEG molecule having a molecular weight of about 20 kDa.

The interferon molecule may be produced according to methods known in the art. Preferably, the interferon molecule is produced recombinantly by expression from a glycosylating host cell (as described in detail in WO 01/15736, PCT/DK02/00128, and in WO 01/36001). The expression host cell may be selected from fungal (filamentous fungal or yeast), insect, mammalian animal cells, from transgenic plant cells or from transgenic animals. Furthermore, the glycosylation may be achieved in the human body when using a nucleotide sequence encoding the polypeptide part of a conjugate of the invention or a polypeptide of the invention in gene therapy. In one embodiment the host cell is a mammalian cell, such as a CHO cell, BHK or HEK cell, e.g. HEK293, or an insect cell, such as an SF9 cell, or a yeast cell, e.g. Saccharomyces cerevisiae, Pichia pastoris or any other suitable glycosylating host. Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Optionally, sugar moieties attached to the polypeptide by in vivo glycosylation are further modified by use of glycosyltransferases, e.g. using the glycoAdvance™ technology marketed by Neose, Horsham, Pa., USA. Thereby, it is possible to, e.g., increase the sialyation of the glycosylated polypeptide following expression and in vivo glycosylation by CHO cells.

The interferon molecule is purified according to methods known in the art to obtain an interferon preparation of sufficient purity to be useful as a drug. Typically, such purification methods involve ultrafiltration, diafiltration, cation exchange chromatography (eg. S-Sepharose from Pharmacia), Hydrofobic Interaction Chromatography, hydroxyapatite chromatography, and/or separation on Sephacryl column.

In another embodiment the interferon molecule is an interferon gamma polypeptide, e.g. wildtype human interferon gamma or a variant thereof, optionally conjugated to a non-polypeptide moiety, such as any of the variants or conjugates described in WO 01/36001.

In a further embodiment of the invention the interferon gamma molecule is selected from an interferon gamma variant, such as any one of those disclosed in the section “Interferon gamma variants” below.

Interferon Gamma Variants

Interferon Gamma Variants with Optimised N-Glycosylation Sites

It has been found that glycosylation of the naturally occurring N-glycosylation site located in position 97 of human interferon gamma may be increased, i.e. an increased fraction of fully, or substantially fully, glycosylated interferon gamma molecules may be obtained, by substituting the serine residue located in position 99 of human interferon gamma with a threonine residue. For example, it has been found that by performing the substitution S99T, about 90% of the interferon gamma polypeptides present in the harvested medium utilized both N-glycosylation site, whereas only about 60% of the recombinantly produced human interferon gamma polypeptides present in the harvested medium was fully glycosylated.

Accordingly, in a very interesting embodiment the interferon gamma polypeptide comprises the substitution S99T.

In addition to the above-mentioned S99T mutation required for optimisation of the in vivo N-glycosylation site at position 97, other in vivo glycosylation sites, which may have been introduced into the sequence (see the section entitled “Interferon gamma variants where the non-polypeptide moiety is a sugar moiety”) may be optimised. Normally, the in vivo glycosylation site is an N-glycosylation site, but also an O-glycosylation site is contemplated as relevant. This optimisation may be achieved by performing a modification, preferably a substitution, in a position, which is located close to a glycosylation site, in particular close to an in vivo N-glycosylation site. Specific examples of suitable positions where in vivo N-glycosylation sites may be introduced, are disclosed in WO 01/36001.

Thus, with respect to the naturally present in vivo N-glycosylation, it is contemplated that the N-glycosylation site at position 97 may be further optimised by performing a modification, such as a substitution, in a position selected from the group consisting of E93, K94, L95, T96, Y98, V100 and T101 (i.e. at position −4, −3, −2, −1, +1, +3 or +4 relative to N97). Specific examples of substitutions performed in position 98 include Y98F, Y98N, Y98Q, Y98V, Y98A, Y98M, Y98I, Y98K, Y98G, Y98R, Y98T, Y98H, Y98C and Y98S, preferably Y98A, Y98M, Y98I, Y98K, Y98G, Y98R, Y98T, Y98H, Y98C and Y98S, in particular Y98S. Specific examples of substitutions performed in position 100 include V100H, V100D, V100A, V100M, V100N, V100T, V100R, V100S, or V100C, in particular V100T, V100R, V100S or V100C.

In a similar way, with respect to the in vivo N-glycosylation site at position 25 it is contemplated that this site may be further optimised by performing a modification, such as a substitution, in a position selected from the group consisting of D21, V22, A23, D24, G26, L28 and F29 (i.e. at position −4, −3, −2, −1, +1, +3 or +4 relative to N25). Specific examples of substitutions performed in position 26 include G26F, G26N, G26Y, G26Q, G26V, G26A, G26M, G26I, G26K, G26R, G26T, G26H, G26C and G26S, preferably G26A, G26M, G26I, G26K, G26R, G26T, G26H, G26C and G26S, more preferably G26A and G26S, in particular G26A. Specific examples of substitutions performed in position 28 include G28H, G28D, G28A, G28M, G28N, G28T, G28R, G28S, or G28S, in particular G28A, G28T, G28R, G28S or G28C.

Obviously, any of the modifications mentioned in connection with optimisation of glycosylation at position 97 may be combined with any of the mentioned in connection with optimisation of glycosylation at position 25.

Interferon Gamma Variants Comprising Attachment Groups for Non-Polypeptide Moieties

Another class of interesting modifications that may be introduced include modifications, which serve to increase the AUC when administered subcutaneously and/or the serum half-life when administered intravenously.

In an interesting embodiment the interferon gamma polypeptide comprises at least one introduced and/or at least one removed amino acid residue comprising an attachment group for a non-polypeptide.

In order to avoid too much disruption of the structure and function of the interferon gamma polypeptide the total number of amino acid residues to be modified in accordance with this embodiment typically does not exceed 15. Usually the amino acid sequence comprises 1-10, such as 1-5 e.g. 1-3 modifications compared to SEQ ID NO:2 (or a C-terminally truncated forms thereof). Preferably, the modification(s) is/are a substitution(s).

In addition to the removal and/or introduction of such amino acid residues, the polypeptide may comprise other modifications, e.g. substitutions, that are not related to introduction and/or removal of amino acid residues comprising an attachment group for the non-polypeptide moiety. Examples of such modifications include conservative amino acid substitutions and/or introduction of Cys-Tyr-Cys or Met at the N-terminus.

The exact number of attachment groups available for conjugation and present in the interferon gamma polypeptide in dimeric form is dependent on the effect desired to be achieved by the conjugation. The effect to be obtained is, e.g., dependent on the nature and degree of conjugation (e.g. the identity of the non-polypeptide moiety, the number of non-polypeptide moieties desirable or possible to conjugate to the polypeptide, where they should be conjugated or where conjugation should be avoided, etc.).

It will be understood that the amino acid residue comprising an attachment group for a non-polypeptide moiety, either it be removed or introduced, is selected on the basis of the nature of the non-polypeptide moiety part of choice and, in most instances, on the basis of the conjugation method to be used. For instance, when the non-polypeptide moiety is a polymer molecule, such as a polyethylene glycol- or polyalkylene oxide-derived molecule, amino acid residues capable of functioning as an attachment group may be selected from the group consisting of cysteine, lysine, aspartic acid, glutamic acid and arginine. In particular, cysteine is preferred. When the non-polypeptide moiety is a sugar moiety the attachment group is, e.g. an in vivo glycosylation site, preferably an N-glycosylation site.

Whenever an attachment group for a non-polypeptide moiety is to be introduced into or removed from the interferon gamma polypeptide, the position of the polypeptide to be modified is conveniently selected as follows:

The position is preferably located at the surface of the interferon gamma polypeptide, and more preferably occupied by an amino acid residue that has more than 25% of its side chain exposed to the solvent, preferably more than 50% of its side chain exposed to the solvent, as determined on the basis of a 3D structure or model of interferon gamma in its dimeric form, the structure or model optionally further comprising one or two interferon gamma receptor molecules. Such positions listed in Example A herein.

In addition, it may be of interest to modify one or more amino acid residues located in the loop regions of interferon gamma since most amino acid residues within these loop regions are exposed to the surface and located sufficiently far away from functional sites so that non-polypeptide moieties, such as polymer molecules, in particular PEG molecules, and/or N-glycosylation sites, may be introduced without impairing the function of the molecule. Such loops regions may be identified by inspection of the three-dimensional structure of human interferon gamma, optinally in complex with its receptor(s). The amino acid residues constituting said loop regions are residues N16-K37 (the “A-B loop”), F60-S65 (the “B-C loop”), N83-S84 (the “C-D loop”) and Y98-L103 (the “D-E loop”).

Furthermore, in the interferon gamma polypeptides, attachment groups located at the receptor-binding site of interferon gamma has preferably been removed, preferably by substitution of the amino acid residue comprising such group. The amino acid residues constituting the interferon gamma receptor-binding site are Q1, D2, Y4, V5, E9, K12, G18, H19, S20, D21, V22, A23, D24, N25, G26, T27, L30, K34, K37, K108, H111, E112, I114, Q115, A118, E119 (see also Example B herein).

In order to determine an optimal distribution of attachment groups, the distance between amino acid residues located at the surface of the interferon gamma polypeptide is calculated on the basis of a 3D structure of the interferon gamma dimeric polypeptide. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In the interferon gamma polypeptide part any of said distances is preferably more than 8 A, in particular more than 10 Å in order to avoid or reduce heterogeneous conjugation.

As mentioned above, under physiological conditions interferon gamma exists as a dimeric polypeptide. The polypeptide is normally in homodimeric form (e.g. prepared by association of two interferon gamma polypeptide molecules prepared as described herein). However, if desired, the interferon gamma polypeptide may be provided in single chain form, wherein two interferon gamma polypeptide monomers are linked via a peptide bond or a peptide linker. Providing the interferon gamma polypeptide in single chain form has the advantage that the two constituent interferon gamma polypeptides may be different which can be advantageous, e.g., to enable asymmetric mutagenesis of the polypeptides. For instance, PEGylation sites can be removed from the receptor-binding site from one of the monomers, but retained in the other. Thereby, after PEGylation one monomer has an intact receptor-binding site, whereas the other may be fully PEGylated (and thus provide significantly increased molecular weight).

Interferon Gamma Variants where the Non-Polypeptide Moiety is a Sugar Moiety

In a preferred embodiment, the interferon gamma polypeptide comprises at least one introduced and/or at least one removed glycosylation site, i.e. the non-polypeptide moiety is a sugar moiety. Preferably, the glycosylation site is an in vivo glycosylation site, i.e. the non-polypeptide moiety is a sugar moiety, e.g. an O-linked or N-linked sugar moiety, preferably an N-linked sugar moiety.

In a particular preferred embodiment the interferon gamma polypeptide comprises at least one introduced glycosylation site, in particular an introduced in vivo N-glycosylation site. Preferably, the introduced glycosylation site is introduced by a substitution.

For instance, an in vivo N-glycosylation site may be introduced into a position of the interferon gamma polypeptide comprising an amino acid residue exposed to the surface. Preferably said surface-exposed amino acid residue has at least 25% of the side chain exposed to the surface, in particular at least 50% of its side chain exposed to the surface. Details regarding determination of such positions can be found in Example A herein.

The N-glycosylation site is introduced in such a way that the N-residue of said site is located in said position. Analogously, an O-glycosylation site is introduced so that the S or T residue making up such site is located in said position. It should be understood that when the term “at least 25% (or 50%) of its side chain exposed to the surface” is used in connection with introduction of an in vivo N-glycosylation site this term refers to the surface accessibility of the amino acid side chain in the position where the sugar moiety is actually attached. In many cases it will be necessary to introduce a serine or a threonine residue in position +2 relative to the asparagine residue to which the sugar moiety is actually attached and these positions, where the serine or threonine residues are introduced, are allowed to be buried, i.e. to have less than 25% (or 50%) of their side chains exposed to the surface of the molecule.

Furthermore, in order to ensure efficient glycosylation it is preferred that the in vivo glycosylation site, in particular the N residue of the N-glycosylation site or the S or T residue of the O-glycosylation site, is located within the 118 N-terminal amino acid residues of the interferon gamma polypeptide, more preferably within the 97 N-terminal amino acid residues. Still more preferably, the in vivo glycosylation site is introduced into a position wherein only one mutation is required to create the site (i.e. where any other amino acid residues required for creating a functional glycosylation site is already present in the molecule).

For instance, substitutions that lead to introduction of an additional N-glycosylation site at positions exposed at the surface of the interferon gamma polypeptide and occupied by amino acid residues having at least 25% of the side chain exposed to the surface (in a structure with receptor molecule) include:

Q1N+P3S/T, P3N+V5S/T, K6N+A8S/T, E9N+L11S/T, K12S/T, K13N+F15S/T, Y14N+N16S/T, G18S/T, G18N, G18N+S20T, H19N+D21S/T, D21N+A23S/T, G26N+L28S/T, G31N+L33S/T, K34N+W36S/T, K37S/T, K37N+E39S/T, E38N, E38N+S40T, E39N+D41S/T, S40N+R42S/T, K55N+F57S/T, K58N+F60S/T, K61S/T, K61N+D63S/T, D62N+Q64S/T, D63N, D63N+S65T, Q64N+166S/T, S65N+Q67S/T, Q67N, Q67N+S69T, K68N+V70S/T, E71N+173S/T, T72N+K74S/T, K74N+D76S/T, E75N+M77S/T, K80S/T, V79N+F81S/T, K80N+F82S/T, N85S/T, S84N+K86S/T, K87S/T, K86N+K88S/T, K87N+R89S/T, D90N+F92S/T, E93N+L95S/T, K94N, K94N+T96S, T101N+L103S/T, D102N+N104S/T, L103N+V105S/T, Q106S/T, E119N, E119N+S121T, P122N+A124S/T, A123N+K125S/T, A124N, A124N+T126S, K125N+G127S/T, T126N+K128S/T, G127N+R129S/T, K128N+K130S/T, R129N+R131 S/T and K130N. S/T indicates a substitution to a serine or threonine residue, preferably a threonine residue.

Substitutions that lead to introduction of an additional N-glycosylation site at positions exposed at the surface of the interferon gamma polypeptide having at least 50% of the side chain exposed to the surface (in a structure with receptor molecule) include:

P3N+V5S/T, K6N+A8S/T, K12S/T, K13N+F15S/T, G18S/T, D21N+A23S/T, G26N+L28S/T, G31N+L33S/T, K34N+W36S/T, K37N+E39S/T, E38N, E38N+S40S/T, E39N+D41S/T, K55N+F57S/T, K58N+F60S/T, K61S/T, D62N+Q64S/T, Q64N+I66S/T, S65N+Q67S/T, K68N+V70S/T, E71N+I73S/T, E75N+M77S/T, N85S/T, S84N+K86S/T, K86N+K88S/T, K87N+R89S/T, K94N, K94N+T96S, T101N+L103S/T, D102N+N104S/T, L103N+V105S/T, Q106S/T, P122N+A124S/T, A123N+K125S/T, A124N, A124N+T126S, K125N+G127S/T, T126N+K128S/T, G127N+R129S/T, K128N+K130S/T, R129N+R131S/T, K130N and K130N+S132T. S/T indicates a substitution to a serine or threonine residue, preferably a threonine residue.

Substitutions where only one amino acid substitution is required to introduce an N-glycosylation site include K12S/T, G18S/T, G18N, K37S/T, E38N, M45N, 149N, K61S/T, D63N, Q67N, V70N, K80S/T, F82N, N85S/T, K87S/T, K94N, Q106S/T, E119N, A124N, K130N and R140N, in particular K12S/T, G18N, G18S/T, K37S/T, E38N, K61 S/T, D63N, Q67N, K80S/T, N85S/T, K94N, Q106S/T, A124N and K130N (positions with more than 25% of its site chain exposed to the surface in a structure without receptor molecule), or more preferably G18N, E38N, D63N, Q67N, K94N, A124N and K130N (positions with more than 50% of its side chain exposed to the surface in a structure without receptor molecule).

Usually, it is not preferred to introduce N-glycosylation sites in the region constituting the receptor binding site (except in special cases, cf. the section entitled “Interferon gamma variants with a reduced receptor affinity”). Accordingly, the mutations Q1N+P3S/T, E9N+L11S/T, G18N, G18N+S20T, H19N+D21 S/T, D21N+A23S/T, G26N+L28S/T, K34N+W36S/T, K37N+E39S/T, E119N and E119N+S121T should normally not be performed, unless a reduced receptor affinity is desired.

Particular preferred interferon gamma polypeptides include at least one substitution selected from the group consisting of K12S, K12T, G18S, G18T, E38N, E38N+S40T, K61S, K61T, N85S, N85T, K94N, Q106S and Q106T, more preferably selected from the group consisting of K12T, G18T, E38N+S40T, K61T, N85T, K94N and Q106T, even more preferably selected from the group consisting of K12T, G18T, E38N+S40T, K61T and N85T, in particular E38N+S40T.

It will be understood that the above-identified substitution are preferably combined with the S99T mutation. Thus, highly preferred interferon gamma polypeptides include substitutions selected from the group consisting of K12S+S99T, K12T+S99T, G18S+S99T, G18T+S99T, E38N+S99T, E38N+S40T+S99T, K61S+S99T, K61T+S99T, N85S+S99T, N85T+S99T, K94N+S99T, S99T+Q106S and S99T+Q106T, more preferably selected from the group consisting of K12T+S99T, G18T+S99T, E38N+S40T+S99T, K61T+S99T, N85T+S99T, K94N+S99T and S99T+Q106T, even more preferably selected from the group consisting of K12T+S99T, G18T+S99T, E38N+S40T+S99T, K61T+S99T and N85T+S99T, in particular E38N+S40T+S99T.

It will be understood that any of the above-mentioned modifications may be combined with any of the modifications disclosed in the section entitled “Interferon gamma variants with optimised N-glycosylation sites”, in particular with the substitution S99T, as well as with any of the modification disclosed in the section entitled “Interferon gamma variants where the non-polypeptide moiety is a molecule, which has cysteine as an attachment group”.

Interferon Gamma Variants where the Non-Polypeptide Moiety is a Molecule, which has Cysteine as an Attachment Group

In another preferred embodiment the interferon gamma polypeptide comprises at least one introduced cysteine residue. Preferably, the cysteine residue is introduced by substitution.

For instance, a cysteine residue may be introduced into a position of the interferon gamma polypeptide comprising an amino acid residue exposed to the surface. Preferably, said surface-exposed amino acid residue has at least 25% of the side chain exposed to the surface, in particular at least 50% of its side chain exposed to the surface. Details regarding determination of such positions can be found in Example A herein.

For instance, substitutions that lead to introduction of a cysteine residue at positions exposed at the surface of the interferon gamma polypeptide and occupied by amino acid residue having at least 25% of the side chain exposed to the surface (in a structure with receptor molecule) include: Q1C, D2C, P3C, K6C, E9C, N10C, K13C, Y14C, N16C, G18C, H19C, D21C, N25C, G26C, G31C, K34C, N35C, K37C, E38C, E39C, S40C, K55C, K58C, N59C, K61C, D62C, D63C, Q64C, S65C, Q67C, K68C, E71C, T72C, K74C, E75C, N78C, V79C, K80C, N83C, S84C, N85C, K86C, K87C, D90C, E93C, K94C, T101C, D102C, L103C, N104C and E119C.

Substitutions that lead to introduction of a cysteine residue at positions exposed at the surface of the interferon gamma polypeptide and occupied by amino acid residue having at least 50% of the side chain exposed to the surface (in a structure with receptor molecule) include: P3C, K6C, N10C, K13C, N16C, D21C, N25C, G26C, G31C, K34C, K37C, E38C, E39C, K55C, K58C, N59C, D62C, Q64C, S65C, K68C, E71C, E75C, N83C, S84C, K86C, K87C, K94C, T101C, D102C, L103C and N104C.

Usually, it is not preferred to introduce cysteine residue (and subsequently attachcing these cysteine residue to a non-polypeptide moiety) in the region constituting the receptor binding site (except in special cases, cf. the section entitled “Interferon gamma variants with a reduced receptor affinity”). Accordingly, the mutations Q1C, E9C, G18C, H19C, D21C, G26C, K34C, K37C and E119C should normally not be performed, unless a reduced receptor affinity is desired.

Most preferably, said cysteine residue is introduced by a substitution selected from the group consisting of N10C, N16C, E38C, N59C, N83C, K94C, N104C and A124C.

Preferably, any of the above-mentioned modified interferon gamma polypeptides further comprises the substitution S99T. Among the above-mentioned substitutions, the following substitutions are particularly preferred N10C+S99T, N16C+S99T, E38C+S99T, N59C+S99T, N83C+S99T, K94C+S99T, N104C+S99T and A124C+S99T.

As will be understood the introduced cysteine residue(s) may preferably be conjugated to a non-polypeptide moiety, such as PEG or more preferably mPEG. The conjugation of the interferon gamma variant and the activated polymer molecules is conducted by use of any conventional method, e.g. as described in the following references (which also describe suitable methods for activation of polymer molecules): Harris and Zalipsky, eds., Poly(ethylene glycol) Chemistry and Biological Applications, AZC, Washington; R. F. Taylor, (1991), “Protein immobilisation. Fundamental and applications”, Marcel Dekker, N. Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.).

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or niPEG polymers will have a size of about 5 kDa, about 10 kD, about 12 kDa or about 20 kDa. For PEGylation to cysteine residues the interferon gamma variant is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

It will be understood that any of the above-mentioned modifications may be combined with any of the modifications disclosed in the section entitled “Interferon gamma variants with optimised N-glycosylation sites”, in particular with the substitution S99T, as well as with any of the modification disclosed in the section entitled “Interferon gamma variants where the non-polypeptide moiety is a sugar moiety”.

Interferon Gamma Variants where a First Non-Polypeptide Moiety is a Sugar Moiety and a Second Non-Polypeptide Moiety is a Molecule, which has Cysteine as an Attachment Group

In a further particular preferred embodiment the interferon gamma polypeptide comprises at least one introduced N-glycosyltion site and at least one introduced cysteine residue. Preferably, the cysteine residue and/or the N-glycosylation site are introduced by substitution. Such polypeptides may be prepared by selecting the residues described in the two preceding sections describing suitable positions for introducing N-glycosylation sites and cysteine residues, respectively. Thus, in an interesting embodiment the interferon gamma polypeptide comprises substitutions selected from the group consisting of K12T+N16C, K12T+E38C, K12T+N59C, K12T+N83C, K12T+K94C, K12T+N104C, K12T+A124C, G18T+N10C, G18T+E38C, G18T+N59C, G18T+N83C, G18T+K94C, G18T+N104C, G18T+A124C, E38N+S40T+N10C, E38N+S40T+N16C, E38N+S40T+N59C, E38N+S40T+N83C, E38N+S40T+K94C, E38N+S40T+N104C, E38N+S40T+A124C, K61T+N10C, K61T+N16C, K61T+E38C, K61T+N83C, K61T+K94C, K61T+N104C, K61T+A124C, N85T+N10C, N85T+N16C, N85T+E38C, N85T+N59C, N85T+K94C, N85T+N104C, N85T+A124C, K94N+N10C, K94N+N16C, K94N+E38C, K94N+N59C, K94N+N83C, K94N+N104C, K94N+A124C, Q106T+N10C, Q106T+N16C, Q106T+E38C, Q106T+N59C, Q106T+N83C, Q106T+K94C and Q106T+A124C, more preferably from the group consisting of E38N+S40T+N10C, E38N+S40T+N16C, E38N+S40T+N59C, E38N+S40T+N83C, E38N+S40T+K94C, E38N+S40T+N104C and E38N+S40T+A124C.

Preferably, any of the above-mentioned modified interferon gamma polypeptides further comprises the substitution S99T, i.e. the interferon gamma polypeptide comprises substitutions selected from the group consisting of K12T+N16C+S99T, K12T+E38C+S99T, K12T+N59C+S99T, K12T+N83C+S99T, K12T+K94C+S99T, K12T+N104C+S99T, K12T+A124C+S99T, G18T+N10C+S99T, G18T+E38C+S99T, G18T+N59C+S99T, G18T+N83C+S99T, G18T+K94C+S99T, G18T+N104C+S99T, G18T+A124C+S99T, E38N+S40T+N10C+S99T, E38N+S40T+N16C+S99T, E38N+S40T+N59C+S99T, E38N+S40T+N83C+S99T, E38N+S40T+K94C+S99T, E38N+S40T+N104C+S99T, E38N+S40T+A124C+S99T, K61 T+N10C+S99T, K61 T+N16C+S99T, K61 T+E38C+S99T, K61 T+N83C+S99T, K61 T+K94C+S99T, K61 T+N104C+S99T, K61 T+A124C+S99T, N85T+N10C+S99T, N85T+N16C+S99T, N85T+E38C+S99T, N85T+N59C+S99T, N85T+K94C+S99T, N85T+N104C+S99T, N85T+A124C+S99T, K94N+N10C+S99T, K94N+N16C+S99T, K94N+E38C+S99T, K94N+N59C+S99T, K94N+N83C+S99T, K94N+N104C+S99T, K94N+A124C+S99T, Q106T+N10C+S99T, Q106T+N16C+S99T, Q106T+E38C+S99T, Q106T+N59C+S99T, Q106T+N83C+S99T, Q106T+K94C+S99T and Q106T+A124C+S99T, more preferably from the group consisting of E38N+S40T+N10C+S99T, E38N+S40T+N16C+S99T, E38N+S40T+N59C+S99T, E38N+S40T+N83C+S99T, E38N+S40T+K94C+S99T, E38N+S40T+N104C+S99T and E38N+S40T+A124C+S99T.

As will be understood, the introduced cysteine residue(s) may preferably be conjugated to a non-polypeptide moiety, such as PEG or more preferably mPEG. The conjugation between the cysteine-containing polypeptide variant and the polymer molecule may be achieved in any suitable manner, e.g. as described in the section entitled “Interferon gamma variants where the non-polypeptide moiety is a molecule, which has cysteine as an attachment group”.

It will be understood that any of the above-mentioned modifications may be combined with any of the modifications disclosed in the section entitled “Interferon gamma variants with optimised in vivo N-glycosylation sites”, in particular with the substitution S99T.

Interferon Gamma Variants with a Reduced Receptor Affinity

One way to increase the serum half-life of an interferon gamma polypeptide would be to decrease the receptor-mediated internalisation and thereby decrease the receptor-mediated clearance. The receptor mediated internalisation is dependent upon the affinity of the interferon gamma dimer for the interferon gamma receptor complex and, accordingly, an interferon gamma polypeptide with a decreased affinity to the interferon gamma receptor complex is expected to be internalised, and hence cleared, to a lesser extent.

The affinity of the interferon gamma dimer to its receptor complex may be decreased by performing one or more modifications, in particular substitutions, in the recpetor binding region of the interferon gamma polypeptide. The amino acid residues which constitute the receptor binding region is defined in Example B herein. One class of substitutions that may be performed is conservative amino acid substitutions. In another embodiment, the modification performed gives rise to the introduction of an N-glycosylation site.

Thus, in a further interesting embodiment the interferon gamma polypeptide comprises at least one modification in the receptor binding site (as defined herein). More particularly, the interferon gamma polypeptide comprises at least one modification, preferably a substitution, which creates an in vivo N-glycosylation site, in said receptor binding region. For instance, such substitutions may be selected from the group consisting of Q1N+P3S/T, D2N+Y4S/T, Y4N+K6S/T, V5N+E7S/T, E9N+L11S/T, K12N+Y14S/T, G18N, G18N+S20T, H19N+D21S/T, S20N+V22S/T, D21N+A23S/T, V22N+D24S/T, D24N+G26S/T, G26N+L28S/T, L30N+I32S/T, K34N+W36S/T, K37N+E39S/T, K108N+1110S/T, H111N+L113S/T, E112N+1114S/T, I114N+V116S/T, Q115N+M117S/T, A118N+L120S/T, E119N and E119N+S121T, preferably from the group consisting of Q1N+P3S/T, D2N+Y4S/T, E9N+L11S/T, K12N+Y14S/T, G18N, G18N+S20T, H19N+D21S/T, S20N+V22S/T, D21N+A23S/T, K34N+W36S/T, K37N+E39S/T, H111N+L 113S/T, Q115N+Ml 17S/T, A118N+L120S/T, E119N and E119N+S121T (introduction of N-glycosylation sites in positions comprising an amino acid residue having at least 25% of its side chain exposed to the surface), more preferably from the group consisting of Q1N+P3S/T, D2N+Y4S/T, E9N+L11S/T, G18N, G18N+S20T, H19N+D21S/T, S20N+V22S/T, D21N+A23S/T, K34N+W36S/T, K37N+E39S/T, Q115N+M117S/T, A118N+L120S/T, E119N and E119N+S121T (introduction of N-glycosylation sites in positions comprising an amino acid residue having at least 50% of its side chain exposed to the surface), even more preferably from the group consisting of Q1N+P3T, D2N+Y4T, E9N+L11T, G18N+S20T, H19N+D21T, S20N+V22T, D21N+A23T, K34N+W36T, K37N+E39T, Q115N+M117T, A118N+L120T and E119N+S121T, most preferably from the group consisting of G18N+S20T, H19N+D21T, D21N+A23T and E 119N+S121T, in particular D21N+A23T.

Such variants are contemplated to exhibit a reduced receptor affinity as compared to human interferon gamma or Actimmune®. The receptor affinity may be measured by any suitable assay and will be known to the person skilled in the art. One example of a suitable assay for determining the receptor binding affinity is the BIAcore® assay described in Michiels et al. Int. J. Biochem. Cell Biol. 30:505-516 (1998).

Typically, such interferon gamma polypeptides having reduced receptor affinity will exhibit a reduced interferon gamma activity, e.g. when tested in the “Primary Assay” described herein. For example, the interferon gamma polypeptide may exhibit 1-95% of the interferon gamma activity of Actimunne® or human interferon gamma, e.g. 1-75%, such as 1-50%, e.g. 1-20% or 1-10% of the interferon gamma activity of Actimunne® or human interferon gamma in its glycosylated form.

Evidently, any of the above-mentioned modifications giving rise to a reduced receptor binding affinity may be combined with any of the other modifications disclosed herein, in particular the modifications mentioned in the sections entitled “Interferon gamma variants with optimised N-glycosylation sites”, “Interferon gamma variants comprising attachment groups for non-polypeptide moieties”, “Inteferon gamma variants where the non-polypeptide moiety is a sugar moiety”, “Interferon gamma variants where the non-polypeptide moiety is a molecule, which has cysteine as an attachment group” and “Interferon gamma variants where a first non-polypeptide moiety is a sugar moiety and a second non-polypeptide moiety is a molecule, which has cysteine as an attachment group”, such as the modifications selected from the group consisiting of E38N, S40T, S99T and combinations thereof, in particular E38N+S40T+S99T.

In a further embodiment the interferon gamma polypeptide comprises at least one introduced glycosylation site, and at least one sugar moiety attached to an introduced glycosylation site. In a further embodiment the interferon gamma polypeptide comprises at least one introduced glycosylation site, and at least one sugar moiety attached to an introduced glycosylation site, and a polymer molecule, such as polyethylene glycol. In a particular preferred embodiment the interferon gamma polypeptide comprises 3 sugar moieties each sugar moiety being attached to an N-glycosylation site.

Analysis of Truncation of Interferon Gamma Polypeptides

Determination of C-terminal truncation of purified samples of interferon gamma polypeptides can be carried out in a number of ways.

One way of elucidating C-terminal truncations of interferon gamma polypeptides relies on accurate mass determinations by mass spectrometry. Unfortunately, the glycosylation of interferon gamma is heterogeneous thus making it extremely difficult to determine an accurate mass directly on the glycoprotein. Therefore, different levels of enzymatic deglycosylation are typically used in combination with mass spectrometry.

In one method, the entire glycan part of the interferon gamma polypeptide is cleaved of using the endo-glycosidase PNGase F followed by accurate mass determination using either ESI mass spectrometry or MALDI-TOF mass spectrometry. Comparing the experimental masses to the known amino acid sequence of interferon gamma makes it possible to determine the sites of C-terminal truncation.

In another related method, only the sialic acid of the glycan part of the interferon gamma polypeptide is cleaved off instead of the entire glycan. In some cases this is sufficient to reduce the heterogeneity of the sample to a level where the sites of C-terminal truncations can be deduced following accurate mass determination using either ESI mass spectrometry or MALDI-TOF mass spectrometry.

A more traditional way of elucidating C-terminal truncations of interferon gamma polypeptides employs peptide mapping in combination with mass spectrometry and chemical amino acid sequencing. In brief, the interferon gamma polypeptide is degraded with a protease of known specificity (e.g. Asp-N protease) followed by peptide separation using RP-HPLC. Fractions can then by mass analysed either on-line using ESI mass spectrometry or off-line using MALDI-TOF mass spectrometry. Comparing the masses obtained for peptides with the known amino acid sequence of interferon gamma makes it possible to determine the likely sites of C-terminal truncation. Verification can then be obtained through amino acid sequencing.

The Sulfoalkyl Ether Cyclodextrin Derivative

In the composition of the invention the sulfoalkyl ether cyclodextrin derivative is any of the derivatives described in U.S. Pat. No. 5,874,418, U.S. Pat. No. 5,376,645 and U.S. Pat. No. 5,134,127, the contents of which are incorporated herein by reference. The sulfoalkyl ether cyclodextrin derivative is also described in WO 91/11172, the contents of which is incorporated herein by reference. In one embodiment of the invention the sulfoalkyl ether cyclodextrin is a compound of the Formula (I):

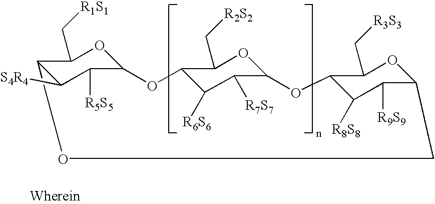

n is 4, 5 or 6,

R₁, R₂, R₃, 4, R₅, R₆, R₇, R₈, and R₉ are each, independently, —O— or a —O—(C₂-C₆ alkylene)-SO₃— group, wherein at least one of R₁ and R₂ is independently a —O—(C₂-C₆ alkylene)-SO₃-group, and

S₁, S₂, S₃, S₄, S₅, S₆, S₇, S₈, and S are each, independently, a pharmaceutically acceptable cation.

In a further embodiment of the compound of the Formula (I) n is 5.

In a further embodiment of the compound of the Formula (I) n is 6.

In a further embodiment of the compound of the Formula (I) at least one of R₁ and R₂ is —O—(CH₂)ₘ—SO₃—, and m is 2, 3, 4, 5 or 6.

In a further embodiment of the compound of the Formula (I) R₁ and R₂ is independently selected from —OCH₂CH₂CH₂SO₃— or —OCH₂CH₂CH₂CH₂SO₃—.

In a further embodiment of the compound of the Formula (I) at least one of R₄, R₆, and R₈, is independently, —O—(C₂-C₆ alkylene)-SO₃—; and R₅, R₇, and R₉ are all —O—.

In a further embodiment of the compound of the Formula (I) S₁, S₂, S₃, S₄, S₅, S₆, S₇, S₈, and S₉ are each, independently, a pharmaceutically acceptable cation selected from H⁺, alkali metals (e.g. Li⁺, Na⁺, K⁺), alkaline earth metals (e.g., Ca⁺², Mg⁺²), ammonium ions and amine cations such as the cations of (C₁-C₆) alkylamines, piperidine, pyrazine, (C₁-C₆) alkanolamine and (C₄-C₈) cycloalkanolamine.

In a further embodiment of the compound of the Formula (I) S₁, S₂, S₃, S₄, S₅, S₆, S₇, S₈, and S₉ are independently selected from alkaline metal cation, alkaline earth metal cation, quaternary ammonium cation, tertiary ammonium cation, and secondary ammonium cation.

In a further embodiment at least one of R₄, R₆, and R₈, is independently, —O—(C₂-C₆ alkylene)-SO₃—; and R₅, R₇, and R₉ are all —O—.

The terms “alkylene” and “alkyl,” as used herein (e.g., in the —O—(C₂-C₆-alkylene)SO₃— group or in the alkylamines), include linear, cyclic, and branched, saturated and unsaturated (i.e., containing one double bond) divalent alkylene groups and monovalent alkyl groups, respectively. The term “alkanol” in this text likewise includes both linear, cyclic and branched, saturated and unsaturated alkyl components of the alkanol groups, in which the hydroxyl groups may be situated at any position on the alkyl moiety. The term “cycloalkanol” includes unsubstituted or substituted (e.g., by methyl or ethyl) cyclic alcohols.

The presently preferred sulfoalkyl ether cyclodextrin derivative is a salt of beta cyclodextrin sulfobutyl ether (in particular the sodium salt thereof also termed SBE7-β-CD which is available as Captisol®) (Cydex, Overland Park, Kans. 66213, US).

Other Embodiments of the Composition of the Invention

Normally, the composition of the invention has a pH in the range of 3-8, such as 4-8, 5-8,6-8, 7-8,4-7, 4-6, or 4-5. For interferon beta, the preferred pH range is 4-8, preferably 5-8, or alternatively 4-7. In a further embodiment the pH range is 5-6, such about 5.5. The pH is normally obtained by use of a suitable amount of a buffering agent as further described below.

Furthermore, it is desirable that the composition is about isotonic to blood, i.e. by having an osmolarity of about 240-360 mOsmol/kg, such as 280-320 mOsmol/kg, in particular about 300 mOsmol/kg. The osmolarity is normally obtained by use of a suitable amount of tonicity agent as further described below.

Accordingly, in a broad aspect the present invention relates to a stabilized composition comprising an interferon molecule and a sulfoalkyl ether cyclodextrin derivative. The interferon molecule is typically selected from any one of those mentioned above and in the examples. Preferred embodiments of the interferon molecule are interferon beta and interferon gamma, in particular interferon beta variants and interferon gamma variants, such as glycosylated and/or pegylated variants, as described in detail above.

The interferon molecule is typically present in a concentration of about 1-100 MIU/ml in a liquid preparation or about 1-100 MIU/dose in a solid preparation. Such concentration may be selected from about 1-10 MIU/ml, 1-20 MIU/ml, 1-30 MIU/ml, 1-40 MIU/ml, 1-50 MIU/ml, 1-60 MIU/ml, 1-70 MIU/ml, 1-80 MIU/ml, 1-90 MIU/ml, 10-20 MIU/ml, 20-30 MIU/ml, 30-40 MIU/ml, 40-50 MIU/ml, 50-60 MIU/ml, 60-70 MIU/ml, 70-80 MIU/ml, 80-90 MIU/ml, 90-100 MIU/ml, 5-95 MIU/ml, 15-85 MIU/ml, 25-75 MIU/ml, 35-65 MIU/ml, and 45-55 MIU/ml in a liquid preparation. Moreover, such concentration may be selected from about 1-10 MIU/dose, 1-20 MIU/dose, 1-30 MIU/dose, 1-40 MIU/dose, 1-50 MIU/dose, 1-60 MIU/dose, 1-70 MIU/dose, 1-80 MIU/dose, 1-90 MIU/dose, 10-20 MIU/dose, 20-30 MIU/dose, 30-40 MIU/dose, 40-50 MIU/dose, 50-60 MIU/dose, 60-70 MIU/dose, 70-80 MIU/dose, 80-90 MIU/dose, 90-100 MIU/dose, 5-95 MIU/dose, 15-85 MIU/dose, 25-75 MIU/dose, 35-65 MIU/dose, and 45-55 MIU/dose in a solid preparation.

The sulfoalkyl ether cyclodextrin derivative is typically present in a concentration of about 1-150 mg/ml. Such concentration may be selected from about 1-10 mg/ml, 1-20 mg/ml, 1-30 mg/ml, 1-40 mg/ml, 1-50 mg/ml, 1-60 mg/ml, 1-70 mg/ml, 1-80 mg/ml, 1-90 mg/ml, 1-100 mg/ml, 1-110 mg/ml, 1-120 mg/ml, 1-130 mg/ml, 1-140 mg/ml, 10-20 mg/ml, 20-30 mg/ml, 30-40 mg/ml, 40-50 mg/ml, 50-60 mg/ml, 60-70 mg/ml, 70-80 mg/ml, 80-90 mg/ml, 90-100 mg/ml, 100-110 mg/ml, 110-120 mg/ml, 120-130 mg/ml, 130-140 mg/ml, 140-150 mg/ml, 5-100 mg/ml, 5-95 mg/ml, 5-90 mg/ml, 5-85 mg/ml, 5-80 mg/ml, 5-75 mg/ml, 5-70 mg/ml, 5-65 mg/ml, 5-60 mg/ml, 5-55 mg/ml, 5-50 mg/ml, 5-45 mg/ml, 5-40 mg/ml, 5-35 mg/ml, 5-30 mg/ml, 5-25 mg/ml, 5-20 mg/ml, 5-15 mg/ml, and 5-10 mg/ml.

In one embodiment, the composition of the invention has a pH in the range of 4-8, and an osmolarity of about 240-360 mOsmol/kg, such as 280-320 mOsmol/kg, in particular about 300 mOsmol/kg.

In a particular aspect the composition of the invention relates to a liquid solution comprising an interferon molecule and a sulfoalkyl ether cyclodextrin derivative.

In a further particular aspect the composition of the invention relates to an aqueous solution comprising an interferon molecule and a sulfoalkyl ether cyclodextrin derivative.

The sulfoalkyl ether cyclodextrin derivative is typically present in a concentration of about 1-150 mg/ml. However, any one of the concentration ranges mentioned above is considered an embodiment of the invention.

The interferon molecule is typically present in a concentration of about 1-100 MIU/ml in the liquid solution. However, any one of the concentration ranges mentioned above is considered an embodiment of the invention.

In an embodiment the liquid solution or aqueous solution is isotonic and has an osmolarity of about 240-360 mOsmol/kg. In a further embodiment the liquid solution or aqueous solution is isotonic and has an osmolarity of about 240-360 mOsmol/kg, and has a pH in the range of 4-8, such as 5-8,6-8, 7-8,4-7, 4-6, or 4-5. In a still further embodiment the liquid solution or aqueous solution further comprises a tonicity agent providing an osmolarity of about 240-360 mOsmol/kg. The tonicity agent may be any suitable tonicity agent such as any one of those mentioned in the section “parenterals” below. In a still further embodiment the liquid solution or aqueous solution further comprises a buffering agent present in a concentration up to 100 mM. The concentration of buffering agent may be selected from any one of the concentration ranges mentioned in the section “parenterals” below. The buffering agent may be any suitable buffer such as any one of those mentioned in the section “parenterals” below.

As mentioned above the interferon molecule is typically selected from any one of those mentioned above and in the examples. Preferred embodiments of the interferon molecule are interferon beta and interferon gamma, in particular any one of the interferon beta variants and interferon gamma variants, such as any one of the glycosylated and/or pegylated variants, as described in detail above. Other preferred embodiments of the interferon molecule are human wild type interferon beta and human wild type interferon gamma, such as glycosylated and/or pegylated molecules, eg. glycosylated interferon beta-1a coupled to a polymer, such as a polymer comprising a polyethylene glycol.

Preferably, the composition of the invention is one, wherein the interferon molecule has essentially retained its bioactivity during storage at a temperature of 37° C. for a period of at least 1 week, preferably at least 2, 3 or 4 weeks (as measured in an accelerated stability test). Alternatively, in the composition of the invention it is preferred that the interferon molecule has essentially retained its bioactivity during storage at a temperature of 25° C. for at least 4 weeks, such as for at least 5, 6, 7, 8, 9, 10, 11 or 12 weeks. In a further alternative, the composition of the invention is one, wherein the interferon molecule has essentially retained its bioactivity during storage at a temperature of 37° C. for a period of at least 1 week, and during storage at a temperature of 25° C. for at least 4 weeks. The bioactivity to be measured can be, for example, any interferon bioactivity described herein, such as antiviral activity, antiproliferative activity, immunomodulatory activity, receptor binding/activation activity, preferably antiviral activity. The term “essentially retained” is intended to mean that the interferon molecule has retained at least 80% of its bioactivity (e.g., antiviral activity) over the course of the test period, preferably at least 85%, more preferably at least 90% or 95%.

The antiviral activity is determined by a method known in the art, e.g. by use of the assays described in WO 01/15736 (Interferon beta) and WO 01/36001 (Interferon gamma).

The assay of WO 01/15736 is as follows:

The antiviral bioassay is performed using A549 cells (CCL 185, American tissue culture collection) and Encephalomyocarditis (EMC) virus (VR-129B, American tissue culture collection). The cells are seeded in 96 well tissue culture plates at a concentration of 10,000 cells/well and incubated at 37° C. in a 5% CO₂ air atmosphere. A polypeptide or conjugate of the invention is added in concentrations ranging from about 100-0.0001 IU/mL (typically from 100-0.0001 IU/mL) in a total of 100 μl DMEM medium containing fetal calf serum and antibiotics. After 24 hours the medium is removed and 0.1 mL fresh medium containing EMC virus is added to each well. The EMC virus is added in a concentration that causes 100% cell death in interferon-beta free cell cultures after 24 hours. After another 24 hrs, the antiviral effect of the polypeptide or conjugate is measured using the WST-1 assay. 0.01 mL WST-1 (WST-1 cell proliferation agent, Roche Diagnostics GmbH, Mannheim, Germany) is added to 0.1 mL culture and incubated for 12-2 hours at 37° C. in a 5% CO₂ air atmosphere The cleavage of the tetrazolium salt WST-1 by mitochondrial dehydrogenases in viable cells results in the formation of formazan that is quantified by measuring the absorbance at 450 nm.

Output is calculated in U/ml relative to a known standard, which is included on the same plate and analyzed under the same conditions.

The assay of WO 01/36001 is as follows:

It has previously been published that interferon gamma interacts with and activates interferon gamma receptors on HeLa cells. Consequently, transcription is activated at promoters containing an Interferon Stimulated Response Element (ISRE). It is thus possible to screen for agonists of interferon receptors by use of an ISRE coupled luciferase reporter gene (ISRE-luc) placed in HeLa cells.

HeLa cells are co-transfected with ISRE-Luc and pcDNA 3.1/hygro and foci (cell clones) are created by selection in DMEM media containing Hygromycin B. Cell clones are screened for luciferase activity in the presence or absence of interferon gamma. Those clones showing the highest ratio of stimulated to unstimulated luciferase activity are used in further assays.

To screen muteins, 15,000 cells/well are seeded in 96 well culture plates and incubated overnight in DMEM media. The next day muteins as well as a known standard are added to the cells in various concentrations. The plates are incubated for 6 hours at 37° C. in a 5% CO₂ air atmosphere LucLite substrate (Packard Bioscience, Groningen The Netherlands) is subsequently added to each well. Plates are sealed and luminescence measured on a TopCount luminometer (Packard) in SPC (single photon counting) mode. Each individual plate contains wells incubated with interferon gamma as a stimulated control and other wells containing normal media as an unstimulated control. The ratio between stimulated and unstimulated luciferase activity serves as an internal standard for both mutein activity and experiment-to-experiment variation.

The pharmaceutical composition of the invention may be in a variety of forms, including liquid, gel, lyophilized, pulmonary dispersion, or any other suitable form, e.g. as a compressed solid. In the present context the term “liquid” is intended to include the term “aqueous”.

In a further embodiment of the invention the composition is in the form of a dry or liquid formulation. In a further embodiment of the invention the composition is in dry form. In a further embodiment of the invention the composition is in liquid form. In a further embodiment of the invention the composition is an aqueous solution. In a further embodiment of the invention the composition is an aqueous suspension.

The preferred form will depend upon the particular indication being treated and will be apparent to one of skill in the art. For instance, lyophilization from a solution may be used to further increase stability.

The pharmaceutical composition of the invention may be administered orally, intravenously, intracerebrally, intramuscularly, intraperitoneally, intradermally, subcutaneously, intranasally, pulmonary, or in any other acceptable manner, e.g. using drug delivery systems like PowderJect or ProLease technology. The preferred mode of administration will depend upon the particular indication being treated and will be apparent to one of skill in the art.

In the following, compositions suitable for specific types of formulations are described. It will be understood that the nature and amount into which various additives are used depend on the interferon molecule as well as the type of formulation and adminstration route. Typically, the composition of the invention comprises a buffering agent, a tonicity agent, a preservative, a wetting agent, a viscosity increasing agent, and/or one or more stabilizers in addition to the sulfoalkyl ether cyclodextrin derivative. It will be understood that such stabilizers must not adversely affect the stabilizing effects of the sulfoalkyl ether cyclodextrin derivative. Additional constituents of the composition are further described below.

Also, the composition of the invention may comprise human serum albumin or other human protein serving to stabilize the composition and/or minimizing adsorption to the container in which the composition is stored. However, in a specific embodiment the composition is essentially free from (i.e., lacks) human serum albumin or other human protein, since the presence of such proteins may be undesirable from a regulatory point of view.

The composition of the invention may be prepared by a conventional method for preparing pharmaceutical compositions. For instance, the composition is prepared by premixing the stabilizing and buffering agents, and any other additives prior to incorporation of the interferon molecule. In one embodiment the composition is prepared with nitrogen purge of aqueous formulation and/or nitrogen purge of void volume of a partly filled product container. In another embodiment the composition is prepared without such nitrogen purge.

The amount of interferon molecule present in the composition depends on the nature of the interferon molecule, of the formulation and of the administration route. For instance, when the composition is an interferon beta containing composition, the interferon beta molecule is present in an amount corresponding to 1-100 MIU/ml, typically 1-50 MIU/ml, (when formulated into a liquid formulation) or 1-100 MIU/dose (when formulated into a solid formulation).

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are prepared for storage as lyophilized formulations or aqueous solutions by mixing, as appropriate, the interferon molecule having the desired degree of purity with one or more pharmaceutically acceptable carriers, excipients or stabilizers typically employed in the art (all of which are termed “excipients”), for example buffering agents, stabilizing agents, preservatives, tonicity agents, non-ionic detergents, antioxidants and/or other miscellaneous additives.

The buffering agent is present in a concentration which ensures that the pH is kept at the desired level, e.g. a level which approximates physiological level. The buffering agents have a suitable concentration up to 100 mM for each buffer type. For most buffering agents this concentration is normally in the range of 1-100 mM, such as 1-90 mM, 1-80 mM, 1-70 mM, 1-60 mM, 1-50 mM, 1-40 mM, 1-30 mM, 1-20 mM, 1-10 mM, 5-15 mM, 15-25 mM, 25-35 mM, 35-45 mM, 45-55 mM, 55-65 mM, 65-75 mM, 75-85 mM, 85-95 mM. A suitable concentration can be determined by the skilled person. The buffering agent is typically a solution of a weak acid, a weak base or a salt of the anion of such acid. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), maleate buffers (eg, sodium maleate), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, carbonate buffers (eg, sodium carbonate), histidine buffers, and glutamate buffers. Preservatives are added to retard microbial growth, and are typically added in amounts of about 0.2%-1% (w/v). Suitable preservatives for use with the present invention include phenol, benzyl alcohol, meta-cresol, alkyl parabens, such as methyl paraben or propyl paraben, benzalkonium halides (e.g. benzalkonium chloride, bromide or iodide), catechol, resorcinol, 3-pentanol and appropriate mixtures thereof.

Thus, in a further embodiment the composition of the invention comprises a buffer, such as any one of the above, or mixtures thereof.

In a further embodiment the composition of the invention further comprises a preservating agent and/or a viscocity increasing agent.

In an alternative embodiment the composition of the invention is free from (i.e., lacks) a preservating agent.

In a further embodiment the composition of the invention comprises a buffer selected from citrate buffers, succinate buffers, tartrate buffers, fumarate buffers, maleate buffers, gluconate buffers, oxalate buffer, phosphate buffers, carbonate buffers, histidine buffers, glutamate buffers, lactate buffers, and acetate buffers.

In a further embodiment the composition of the invention comprises a buffer which is present in a concentration up to 100 mM, such as 1 mM to 100 mM, 1-90 mM, 1-80 mM, 1-70 mM, 1-60 mM, 1-50 mM, 1-40 mM, 1-30 mM, 1-20 mM, 1-10 mM, 5-15 mM, 15-25 mM, 25-35 mM, 35-45 mM, 45-55 mM, 55-65 mM, 65-75 mM, 75-85 mM, or 85-95 mM.

Tonicity agents are added to ensure isotonicity of liquid compositions and include polyhydric sugar alcohols, preferably trihydric or higher sugar alcohols, such as glycerin, erythritol, arabitol, xylitol, sorbitol, and mannitol (mannitol is typically present in a concentration of up to 50 mg/ml), or NaCl (NaCl is typically present in a concentration of up to 9 mg/ml). Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® is polyols, polyoxyethylene sorbitan monoethers (Tween®-20 (Tween-20 is typically present in a concentration of up to 2 mg/ml), Tween®-80 (Tween-80 is typically present in a concentration of up to 2 mg/ml), etc.).

In a preferred embodiment no surfactant, such as non-ionic surfactant, is added when the composition comprises an interferon beta molecule.

Additional miscellaneous excipients include bulking agents or fillers (e.g. starch), chelating agents (e.g. EDTA), antioxidants (e.g., ascorbic acid, methionine, vitamin E) and cosolvents. The active ingredient may also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in sustained release preparations (see next section). Such techniques are disclosed in Remington's Pharmaceutical Sciences, by E. W. Martin, 18th edition, A. R. Gennaro, Ed., Mack Publishing Company [1990]; Pharmaceutical Formulation Development of Peptides and Proteins.

Parenteral formulations to be used for in vivo administration must be sterile. This is readily accomplished, for example, by filtration through sterile filtration membranes.

Sustained Release Preparations (for Paranteral or Other Use)

Suitable examples of sustained-release preparations include semi-permeable matrices of solid hydrophobic polymers and/or hydrophilic polymers containing the interferon molecule, the matrices having a suitable form such as a film, a rod, a microcapsule or a microsphere. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, dextrans, starch, degradable lactic acid-glycolic acid copolymers such as the ProLease® technology or Lupron Depot® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release proteins for shorter time periods.

Pulmonary or Nasal Delivery Formulations

Such formulations may be in the form of liquid or solid formulations to be used as is or in the form of a dispersion.

All relevant devices for administration and/or generation of a dispersion require the use of formulations suitable for dispensing the interferon molecule. Typically, each formulation is specific to the type of device employed and may involve the use of an appropriate propellant material, in addition to the usual diluents, adjuvants and/or carriers useful in therapy. Each formulation is also typically specific to the interferon molecule to be delivered as a dispersion. Also, the use of liposomes, microcapsules or microspheres, inclusion complexes, or other types of carriers is contemplated. Formulations of the interferon molecule which can be utilized in the most common types of pulmonary dispensing devices to practice this invention are now described.

Nebulizer Interferon Formulation

Interferon molecule formulations suitable for use with a nebulizer, either jet or ultrasonic, will typically comprise the interferon molecule dissolved in water at a concentration of, e.g., about 0.01 to 25 mg of interferon molecule per ml of solution, preferably about 0.1 to 10 mg/ml. The formulation may also include a buffer and a tonicity agent, e.g. a sugar for protein stabilization and regulation of osmotic pressure, and/or human serum albumin ranging in concentration from 0.1 to 10 mg/ml. Examples of buffers which may be used are sodium acetate, citrate and glycine. Preferably, the buffer will have a composition and molarity suitable to adjust the solution to a pH in the range of 3 to 9. Generally, buffer molarities of from 1 mM to 50 mM are suitable for this purpose. Examples of sugars which can be utilized are lactose, maltose, mannitol, sorbitol, trehalose, and xylose, usually in amounts ranging from 1% to 10% by weight of the formulation.

The nebulizer formulation may also contain a surfactant to reduce or prevent surface induced aggregation of the protein caused by atomization of the solution in forming the aerosol. Various conventional surfactants can be employed, such as polyoxyethylene fatty acid esters and alcohols, and polyoxyethylene sorbitan fatty acid esters. Amounts will generally range between 0.001% and 4% by weight of the formulation. An especially preferred surfactant for purposes of this invention is polyoxyethylene sorbitan monooleate.

Specific formulations and methods of generating suitable dispersions of liquid particles are described in WO 9420069, U.S. Pat. No. 5,915,378, U.S. Pat. No. 5,960,792, U.S. Pat. No. 5,957,124, U.S. Pat. No. 5,934,272, U.S. Pat. No. 5,915,378, U.S. Pat. No. 5,855,564, U.S. Pat. No. 5,826,570 and U.S. Pat. No. 5,522,385 which are hereby incorporated by reference.

Three specific examples of commercially available nebulizers suitable for the practice of this invention are the Ultravent nebulizer, manufactured by Mallinckrodt, Inc., St. Louis, Mo., the Acorn II nebulizer, manufactured by Marquest Medical Products, Englewood, Colo., and the AERx pulmonary drug delivery system manufactured by Aradigm Corporation, Hayward, Calif.

Dry Formulation for Metered Dose Inhalers and Powder Inhalers

Interferon formulations for use with a metered dose inhaler device will generally comprise a finely divided powder of relevant shape, surface and size. This powder may be produced by lyophilizing and, if needed, then milling a solid interferon formulation and may also contain a stabilizer such as human serum albumin (HSA). Typically, more than 0.5% (w/w) HSA is added. Additionally, one or more sugars or sugar alcohols may be added to the preparation if necessary. Examples include lactose maltose, mannitol, sorbitol, sorbitose, trehalose, xylitol, and xylose. The amount added to the formulation can range from about 0.01 up to 100% (w/w), preferably from approximately 1 to 50%. Such formulations are then lyophilized and milled to the desired particle size.

The properly sized particles are then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. An example of a commercially available metered dose inhaler suitable for use in the present invention is the Ventolin metered dose inhaler, manufactured by Glaxo Inc., Research Triangle Park, N.C.

Such interferon formulations for powder inhalers will comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder shall have aerodynamic properties in the lung. This typically corresponds to particles with a density of about 1 g/cm2 having a median diameter less than 10 micrometers, preferably between 0.5 and 5 micrometers, most preferably of between 1.5 and 3.5 micrometers.

An example of a powder inhaler suitable for use in accordance with the teachings herein is the Spinhaler powder inhaler, manufactured by Fisons Corp., Bedford, Mass.

The powders for these devices may be generated and/or delivered by methods disclosed in U.S. Pat. No. 5,997,848, U.S. Pat. No. 5,993,783, U.S. Pat. No. 5,985,248, U.S. Pat. No. 5,976,574, U.S. Pat. No. 5,922,354, U.S. Pat. No. 5,785,049 and U.S. Pat. No. 5,565,4007 which are hereby incorporated by reference.

Mechanical Devices for the Administration of the Dispersion of the Invention

The pharmaceutical composition containing the interferon molecule may be administered by a wide range of mechanical devices designed for pulmonary delivery of therapeutic products, including but limited to nebulizers, metered dose inhalers, and powder inhalers, all of which are familiar to those of skill in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; the Spinhaler powder inhaler, manufactured by Fisons Corp., Bedford, Mass.; the “standing cloud” device of Inhale Therapeutic Systems, Inc., San Carlos, Calif.; the AIR inhaler manufactured by Alkermes, Cambridge, Mass.; and the AERx pulmonary drug delivery system manufactured by Aradigm Corporation, Hayward, Calif.

The present invention also provides for a primary product container comprising a composition of the invention. The container may be any type of container suited for the composition in question, e.g. made from stainless steel or glass. The container may be a syringe, such as a prefilled syringe.

Furthermore, the invention provides for a kit for parental administration of a liquid interferon composition according to the invention, and instructions for use. The kit may comprise the interferon composition as the only pharmaceutically active ingredient, or may comprise one or more further pharmaceutically active ingredients—either in the same container or a separate container.

In a further aspect the invention relates to a method for increasing stability of an interferon molecule formulated into a pharmaceutical composition, said method comprising incorporating into said composition a sulfoalkyl ether cyclodextrin derivative and a buffering agent.

The method is of particular relevance when the interferon molecule exhibits aggregate formation during storage and the sulfoalkyl ether cyclodextrin derivative is incorporated in an amount sufficient to reduce aggregate formation of the interferon molecule. The interferon molecule is preferably any of those described herein. The sulfoalkyl ether cyclodextrin derivative is preferably any of those described in U.S. Pat. No. 5,376,645, U.S. Pat. No. 5,874,418 or U.S. Pat. No. 5,134,127, in particular a salt of beta cyclodextrin sulfobutyl ether, e.g. the sodium salt available as Captisol®.

In a still further aspect the invention relates to a method of subjecting a mammal to interferon therapy, which method comprises administering a therapeutically effective amount of a composition according to the invention. Also, the invention relates to a composition of the invention for use in treatment of diseases as well as to the use of a composition of the invention for the manufacture of a medicament for treatment of diseases. It is clear that when a composition of the invention is used as a medicine in the treatment of a disease, disorder, or condition, it is a pharmaceutical composition.

The pharmaceutical composition of the invention may be administered in conjunction with other therapeutic agents. These agents may be incorporated as part of the same pharmaceutical composition or may be administered separately from the interferon molecule either concurrently or in accordance with any other acceptable treatment schedule. In addition, the pharmaceutical composition of the invention may be used as an adjunct to other therapies.

It will be understood that the disease, disorder, or condition to be treated depends on the type of interferon molecule present in the composition.

When the interferon molecule is interferon alpha, or interferon beta, or a variant or a conjugate thereof, this invention provides compositions and methods for treating a disease, disorder, or condition for which interferon alpha or interferon beta is a useful treatment or may potentially be a useful treatment, such as, for example: most types of viral infections, cancers or tumors or tumour angiogenesis, Chrohn's disease, ulcerative colitis, Guillain-Barré syndrome, glioma, idiopathic pulmonary fibrosis, abnormal cell growth, or for immunomodulation in any suitable animal, preferably mammal, and in particular human. For example, the composition of the invention may be used in the treatment of osteosarcoma, basal cell carcinoma, ovarian carcinoma, cervical dysplasia, cervical carcinoma, laryngeal papillomatosis, mycosis fungoides, glioma, acute myeloid leukemia, multiple myeloma, Hodgkin's disease, melanoma, breast carcinoma, non-small cell lung cancer, malignant melanoma (adjuvant, late stage, as well as prophylactic), carcinoid tumour, B-cell lymphoma, T-cell lymphoma, follicular lymphoma, Kaposi's sarcoma, chronic myelogenous leukaemia, renal cell carcinoma, recurrent superfiecial bladder cancer, colorectal carcinoma, hairy cell leukaemia, and viral infections such as papilloma virus, viral hepatitis, herpes genitalis, herpes zoster, herpetic keratitis, herpes simplex, viral encephalitis, cytomegalovirus pneumonia, rhinovirus chronic persistent hepatitis, chronic active HCV (type I), chronic active HCV (type II) and chronic hepatitis B.

In this connection, the composition may be used for CML monotherapy or in combination with cytarabne, for B-cell lymphoma monotherapy or in combination with doxorubicin-based regimens, for follicular lymphoma therapy as an adjunct to CHOP-like regimen, for hepatitis C monotherapy or in combination with ribavirin, for multiple myeloma monotherapy or in combination with VBMCP, BCNU or VBMCP+HiCy, or for renal carcinoma monotherapy or in combination with Vinblastine, floxuridine, 5-fluoruouracil or IL-10.

In particular the molecule or composition of the invention may be used for the treatment of multiple sclerosis (MS), such as any of the generally recognized four types of MS (benign, relapsing remitting MS (RRMS), primary progressive MS (PPMS) and secondary progressive MS (SPMS)) and for monosymptomatic MS), cancer or tumours, hepatitis, e.g. hepatitis B and hepatitis C, or a herpes infection (the latter treatment optionally being combined with a treatment with IL-10).

Thus, the present invention also relates to use of a composition comprising an interferon beta molecule and a sulfoalkyl ether cyclodextrin derivative for the manufacture of a medicament for treatment of diseases, disorders, or conditions for which interferon beta is a useful treatment or may potentially be a useful treatment, such as, for example: viral infections, cancers or tumors or tumour angiogenesis, Crohn's disease, ulcerative colitis, Guillain-Barré syndrome, glioma, idiopathic pulmonary fibrosis, abnormal cell growth, or for immunomodulation in any suitable animal, preferably mammal, and in particular human. More specifically the composition of the invention comprising an interferon beta molecule and a sulfoalkyl ether cyclodextrin derivative may be used in the treatment of osteosarcoma, basal cell carcinoma, ovarian carcinoma, cervical dysplasia, cervical carcinoma, laryngeal papillomatosis, mycosis fungoides, glioma, acute myeloid leukemia, multiple myeloma, Hodgkin's disease, melanoma, breast carcinoma, non-small cell lung cancer, malignant melanoma (adjuvant, late stage, as well as prophylactic), carcinoid tumour, B-cell lymphoma, T-cell lymphoma, follicular lymphoma, Kaposi's sarcoma, chronic myelogenous leukaemia, renal cell carcinoma, recurrent superfiecial bladder cancer, colorectal carcinoma, hairy cell leukaemia, and viral infections such as papilloma virus, viral hepatitis, herpes genitalis, herpes zoster, herpetic keratitis, herpes simplex, viral encephalitis, cytomegalovirus pneumonia, rhinovirus chronic persistent hepatitis, chronic active HCV (type I), chronic active HCV (type II), chronic hepatitis B, chronic or acute hepatitis C, or a herpes infection, multiple sclerosis (MS), such as any of the generally recognized four types of MS (benign, relapsing remitting MS (RRMS), primary progressive MS (PPMS) and secondary progressive MS (SPMS)) and for monosymptomatic MS).

In a particular aspect, the present invention relates to use of a composition comprising an interferon beta molecule and a sulfoalkyl ether cyclodextrin derivative, wherein the interferon beta molecule comprises the substitutions C17S+Q49N+Q51T+D110F+F111N+R113T relative to the wildtype human interferon beta shown in SEQ ID NO:1, and has 3 sugar moieties attached to 3 N-glycosylation sites and 1 PEG molecule having a molecular weight of about 20 kDa (a particular example of such an interferon beta molecule is eg. the interferon beta variant of example 3 pegylated with a 20 kDa-PEG), for the manufacture of a medicament for treatment of a cancer selected from any one of osteosarcoma, basal cell carcinoma, ovarian carcinoma, cervical dysplasia, cervical carcinoma, laryngeal papillomatosis, mycosis fungoides, glioma, acute myeloid leukemia, multiple myeloma, Hodgkin's disease, melanoma, breast carcinoma, non-small cell lung cancer, malignant melanoma (adjuvant, late stage, as well as prophylactic), carcinoid tumour, B-cell lymphoma, T-cell lymphoma, follicular lymphoma, Kaposi's sarcoma, chronic myelogenous leukaemia, renal cell carcinoma, recurrent superfiecial bladder cancer, colorectal carcinoma, and hairy cell leukaemia. Each of the above cancers is considered an embodiment of the present invention. In a preferred embodiment the cancer is melanoma. In another preferred embodiment the cancer is breast carcinoma. In a further preferred embodiment the cancer is non-small cell lung cancer. In a further preferred embodiment the cancer is malignant melanoma.

Thus, the present invention also relates to use of a composition comprising an interferon alfa molecule and a sulfoalkyl ether cyclodextrin derivative for the manufacture of a medicament for treatment of diseases, disorders, or conditions for which interferon alpha is a useful treatment or may potentially be a useful treatment, such as, for example: viral infections, cancers or tumors or tumour angiogenesis, Crohn's disease, ulcerative colitis, Guillain-Barré syndrome, glioma, idiopathic pulmonary fibrosis, abnormal cell growth, or for immunomodulation in any suitable animal, preferably mammal, and in particular human. More specifically the composition of the invention comprising an interferon alfa molecule and a sulfoalkyl ether cyclodextrin derivative may be used in the treatment of osteosarcoma, basal cell carcinoma, ovarian carcinoma, cervical dysplasia, cervical carcinoma, laryngeal papillomatosis, mycosis fungoides, glioma, acute myeloid leukemia, multiple myeloma, Hodgkin's disease, melanoma, breast carcinoma, non-small cell lung cancer, malignant melanoma (adjuvant, late stage, as well as prophylactic), carcinoid tumour, B-cell lymphoma, T-cell lymphoma, follicular lymphoma, Kaposi's sarcoma, chronic myelogenous leukaemia, renal cell carcinoma, recurrent superfiecial bladder cancer, colorectal carcinoma, hairy cell leukaemia, and viral infections such as papilloma virus, viral hepatitis, herpes genitalis, herpes zoster, herpetic keratitis, herpes simplex, viral encephalitis, cytomegalovirus pneumonia, rhinovirus chronic persistent hepatitis, chronic active HCV (type I), chronic active HCV (type II), chronic hepatitis B, chronic or acute hepatitis C, or a herpes infection, multiple sclerosis (MS), such as any of the generally recognized four types of MS (benign, relapsing remitting MS (RRMS), primary progressive MS (PPMS) and secondary progressive MS (SPMS)) and for monosymptomatic MS).

Also, when the composition comprises an interferon beta molecule the invention relates to a method of treating a mammal having circulating antibodies against interferon beta 1a, such as Avonex™ or Rebif®, or 1b, such as Betaseron®, which method comprises administering a composition of the invention comprising an interferon beta molecule which has a reduced or no reaction with said antibodies. The compound is administered in an effective amount. The mammal is preferably a human being. The mammals to be treated may suffer from any of the diseases, disorders, or conditions listed above for which interferon β is a useful treatment or may potentially be a useful treatment. In particular, this aspect of the invention is of interest for the treatment of multiple sclerosis (any of the types listed above), hepatitis or cancer. The term “circulating antibodies” is intended to indicate antibodies, in particular neutralizing antibodies, formed in a mammal in response to having been treated with any of the commercially available interferon beta preparations (Rebif, Betaseron, Avonex).

When the interferon molecule is interferon gamma or a variant or conjugate thereof, the composition of the invention may be used for treatment of a disease, disorder, or condition for which interferon gamma is a useful treatment or may potentially be a useful treatment, such as, for example, any of the medical indications described in WO 01/36001, in particular interstitial pulmonary diseases, most particularly idiopathic pulmonary fibrosis. Interferon gamma has been suggested for treatment of interstitial lung diseases (also known as Interstitial Pulmonary Fibrosis (IPF) (Ziesche et al. (N. Engl. J. Med. 341:1264-1269, 1999 and Chest 110:Suppl:25S, 1996) and EP 795332) for which purpose interferon gamma can be used in combination with prednisolone. In addition to IPF, granulomatous diseases (Bolinger et al, Clinical Pharmacy, 1992, 11:834-850), certain mycobacterial infections (N. Engl. J. Med. 330:1348-1355, 1994), kidney cancer (J. Urol. 152:841-845, 1994), osteopetrosis (N. Engl. J. Med. 332:1594-1599, 1995), scleroderma (J. Rheumatol. 23:654-658, 1996), hepatitis B (Hepatogastroenterology 45:2282-2294, 1998), hepatitis C (Int. Hepatol. Communic. 6:264-273, 1997), septic shock (Nature Medicine 3:678-681, 1997), and rheumatoid arthritis may be treated with interferon gamma.

Thus, the present invention also relates to use of a composition comprising an interferon gamma molecule and a sulfoalkyl ether cyclodextrin derivative for the manufacture of a medicament for treatment of diseases, disorders, or conditions for which interferon gamma is a useful treatment or may potentially be a useful treatment, such as, for example: interstitial pulmonary diseases, most particularly idiopathic pulmonary fibrosis, interstitial lung diseases (also known as Interstitial Pulmonary Fibrosis (IPF)), granulomatous diseases, mycobacterial infections, kidney cancer, osteopetrosis, scleroderma, hepatitis B, hepatitis C, septic shock, and rheumatoid arthritis.

### EXAMPLES

Materials and Methods for Preparing Interferon Gamma

Materials

CHO-K1 cells (available from American Type Culture Collection (ATCC #CCL-61)).

HeLa cells (available from American Type Culture Collection (ATCC #CCL-2)).

ISRE-Luc was obtained from Stratagene, La Jolla USA.

pcDNA 3.1/hygro was obtained from Invitrogen, Carlsbad USA.

Restricion enzymes and polymerases were obtained from New England Biolabs Inc., Beverly, USA.

DMEM medium: Dulbecco's Modified Eagle Media (DMEM), 10% fetal bovine serum and Hygromycin B were obtained from Life Technologies A/S, Copenhagen, Denmark.

LucLite substrate was obtained from Packard Bioscience, Groningen, The Netherlands.

TopCount luminometer was obtained from Packard Bioscience, Groningen, The Netherlands.

Biotinylated polyclonal anti-human interferon gamma antibody, BAF285, was obtained available from R&D Systems Inc., Minneapolis, USA.

Horse Radish Peroxidase-conjugated streptavidin, P0397, was obtained from DAKO, Copenhagen, Denmark.

TMB blotting reagent was obtained from KEM-EN-TEC, Copenhagen, Denmark.

Methods

Interferon Assay Outline

It has previously been published that interferon gamma interacts with and activates interferon gamma receptors on HeLa cells. Consequently, transcription is activated at promoters containing an Interferon Stimulated Response Element (ISRE). It is thus possible to screen for agonists of interferon receptors by use of an ISRE coupled luciferase reporter gene (ISRE-luc) placed in HeLa cells.

Primary Assay

HeLa cells are co-transfected with ISRE-Luc and pcDNA 3.1/hygro and foci (cell clones) are created by selection in DMEM media containing Hygromycin B. Cell clones are screened for luciferase activity in the presence or absence of interferon gamma. Those clones showing the highest ratio of stimulated to unstimulated luciferase activity are used in further assays.

To screen polypeptides, 15,000 cells/well are seeded in 96 well culture plates and incubated overnight in DMEM media. The next day the polypeptides as well as a known standard are added to the cells in various concentrations. The plates are incubated for 6 hours at 37° C. in a 5% CO₂ air atmosphere LucLite substrate (Packard Bioscience, Groningen, The Netherlands) is subsequently added to each well. Plates are sealed and luminescence measured on a TopCount luminometer (Packard) in SPC (single photon counting) mode.

Each individual plate contains wells incubated with interferon gamma as a stimulated control and other wells containing normal media as an unstimulated control. The ratio between stimulated and unstimulated luciferase activity serves as an internal standard for both interferon gamma activity and experiment-to-experiment variation.

Identification of Surface Exposed Amino Acid Residues

Structures

Experimental 3D structures of human interferon gamma determined by X-ray crystallography have been reported by: Ealick et al. Science 252:698-702 (1991) reporting on the C-alpha trace of an interferon gamma homodimer. Walter et. al. Nature 376:230-235 (1995) reporting on the structure of an interferon gamma homodimer in complex with two molecules of a soluble form of the interferon gamma receptor. The coordinates of this structure have never been made publicly available. Thiel et. al. Structure 8:927-936 (2000) reporting on the structure of an interferon gamma homodimer in complex with two molecules of a soluble form of the interferon gamma receptor having a third molecule of the receptor in the structure not making interactions with the interferon gamma homodimer.

Accessible Surface Area (ASA)

The computer program Access (B. Lee and F. M. Richards, J. Mol. Biol. 55: 379-400 (1971)) version 2 (Copyright (c) 1983 Yale University) was used to compute the accessible surface area (ASA) of the individual atoms in the structure. This method typically uses a probe-size of 1.4A and defines the Accessible Surface Area (ASA) as the area formed by the centre of the probe. Prior to this calculation all water molecules, hydrogen atoms and other atoms not directly related to the protein are removed from the coordinate set.

Fractional ASA of Side Chain

The fractional ASA of the side chain atoms is computed by division of the sum of the ASA of the atoms in the side chain with a value representing the ASA of the side chain atoms of that residue type in an extended ALA-x-ALA tripeptide. See Hubbard, Campbell & Thornton (1991) J. Mol. Biol.: 220,507-530. For this example the CA atom is regarded as a part of the side chain of Glycine residues but not for the remaining residues. The following table are used as standard 100% ASA for the side chain:

| | | | | | |
|---|---|---|---|---|---|
| Ala | 69.23 | Å² | Leu | 140.76 | Å² |
| Arg | 200.35 | Å² | Lys | 162.50 | Å² |
| Asn | 106.25 | Å² | Met | 156.08 | Å² |
| Asp | 102.06 | Å² | Phe | 163.90 | Å² |
| Cys | 96.69 | Å² | Pro | 119.65 | Å² |
| Gln | 140.58 | Å² | Ser | 78.16 | Å² |
| Glu | 134.61 | Å² | Thr | 101.67 | Å² |
| Gly | 32.28 | Å² | Trp | 210.89 | Å² |
| His | 147.00 | Å² | Tyr | 176.61 | Å² |
| Ile | 137.91 | Å² | Val | 114.14 | Å² |

Residues not detected in the structure are defined as having 100% exposure as they are thought to reside in flexible regions.

Determining Distances Between Atoms:

The distance between atoms was determined using molecular graphics software e.g. InsightII v. 98.0, MSI INC.

Determination of Receptor Binding Site:

The receptor-binding site is defined as comprising of all residues having their accessible surface area changed upon receptor binding. This is determined by at least two ASA calculations; one on the isolated ligand(s) in the ligand(s)/receptor(s) complex and one on the complete ligand(s)/receptor(s) complex.

### Example A

### Determination of Surface-Exposed Amino Acids

The X-ray structure used was of an interferon gamma homo-dimer in complex with two molecules of a soluble form of the interferon gamma receptor having a third molecule of the interferon gamma receptor in the structure not making interactions with the interferon gamma homodimer reported by Thiel et. al. Structure 8:927-936 (2000). The structure consists of the interferon gamma homodimer wherein the two molecules are labeled A and B. For construction purposes there is an additional methionine placed before the interferon gamma sequence labeled M0 and the sequence is C-terminally truancuted with ten residues (Q133 being the last residue in the constructed molecules). The M0 is removed from the structure in all the calculations of this example. The structure of the two interferon gamma monomers has very weak electron density after residue 120 and residues were only modeled until residue T126. Therefore, residues S121-T126 were removed from the structure prior to the calculations in this example. The two receptor fragments labeled C and D make direct interactions with the interferon gamma homodimer and a third receptor molecule labeled E makes no contact with the interferon gamma homodimer and are not included in these calculations.

Surface Exposure:

Performing fractional ASA calculations on the homodimer of molecules A and B excluding M0 and S121-T126 in both molecules resulted in the following residues having more than 25% of their side chain exposed to the surface in at least one of the monomers: Q1, D2, P3, K6, E9, N10, K12, K13, Y14, N16, G18, H19, S20, D21, A23, D24, N25, G26, T27, G31, K34, N35, K37, E38, E39, S40, K55, K58, N59, K61, D62, D63, Q64, S65, Q67, K68, E71, T72, K74, E75, N78, V79, K80, N83, S84, N85, K86, K87, D90, E93, K94, N97, S99, T101, D102, L103, N104, H111, Q15, A118 and E 119.

The following residues had more than 50% of their side chain exposed to the surface in at least one of the monomers: Q1, D2, P3, K6, E9, N10, K13, N16, G18, H19, S20, D21, A23, D24, N25, G26, T27, G31, K34, K37, E38, E39, K55, K58, N59, D62, Q64, S65, K68, E71, E75, N83, S84, K86, K87, K94, N97, S99, T101, D102, L103, N104, Q115, A 118, E119.

Performing fractional ASA calculations on the homodimer of molecules A and B excluding M0 and S121-T126 in both molecules and including the receptor molecules C and D resulted in the following residues had more than 25% of their side chain exposed to the surface in at least one of the monomers: Q1, D2, P3, K6, E9, N10, K13, Y14, N16, G18, H19, D21, N25, G26, G31, K34, N35, K37, E38, E39, S40, K55, K58, N59, K61, D62, D63, Q64, S65, Q67, K68, E71, T72, K74, E75, N78, V79, K80, N83, S84, N85, K86, K87, D90, E93, K94, N97, S99, T101, D102, L103, N104, E 119.

The following residues had more than 50% of their side chain exposed to the surface in at least one of the monomers: P3, K6, N10, K13, N16, D21, N25, G26, G31, K34, K37, E38, E39, K55, K58, N59, D62, Q64, S65, K68, E71, E75, N83, S84, K86, K87, K94, N97, S99, T101, D102, L103 and N104.

Comparing the two lists, results in K12, S20, A23, D24, T27, H111, Q115 and A 118 being removed from the more than 25% side chain ASA list upon receptor binding, and Q1, D2, E9, G18, H19, S20, A23, D24, T27, Q115, A 118 and E 119 being removed from the more than 50% side chain ASA list upon receptor binding.

Residues not determined in the structure are treated as fully surface exposed, i.e. residues S121, P122, A123, A124, K125, T126, G127, K128, R129, K130, R131, S132, Q133, M134, L135, F136, R137, G138, R139, R140, A141, S142, Q143. These residues also constitute separate targets for introduction of attachment groups (or may be viewed as belonging to the group of surface exposed amino acid residues, e.g. having more than 25% or more than 50% exposed side chains).

### Example B

### Determination of Receptor Binding Site

Performing ASA calculations as described above results in the following residues of the interferon gamma molecule having reduced ASA in at least one of the monomers in the complex as compared to the calculation on the isolated dimer: Q1, D2, Y4, V5, E9, K12, G18, H19, S20, D21, V22, A23, D24, N25, G26, T27, L30, K34, K37, K108, H111, E 112, 1114, Q115, A 118, E 119.

### Example C

### Design of an Expression Cassette for Expression of Interferon Gamma with Codon Usage Optimised for CHO Cells

The DNA sequence, GenBank accession number X13274, encompassing a full length cDNA encoding mature human interferon gamma with its native signal peptide, was modified in order to facilitate high expression in CHO cells. Codons of the human interferon gamma nucleotide sequence were modified by making a bias in the codon usage towards the codons frequently used in homo sapiens. Subsequently, certain nucleotides in the sequence were substituted with others in order to introduce recognition sites for DNA restriction endonucleases. Primers were designed such that the gene could be synthesised.

The primers were assembled to the synthetic gene by one step PCR using Platinum Pfx-polymerase kit (Life Technologies) and standard three-step PCR cycling parameters. The assembled gene was amplified by PCR using the same conditions and has the sequence shown in SEQ ID NO:5. The synthesised gene was cloned into pcDNA3.1/hygro (InVitrogen) between the BamHI at the 5′ end and the XbaI at the 3′ end, resulting in pIGY-22.

### Example D

### Site Directed Mutagenesis Generation of Glycosylation Variants

To introduce mutations in interferon gamma, oligonucleotides were designed in such a way that PCR-generated changes could be introduced in the expression plasmid (pIGY-22) by classical two-step PCR.

Two vector primers were used together with specific mutation primers: ADJ013: 5′-GATGGCTGGCAACTAGAAG-3′ (SEQ ID NO:6), (antisense downstream vector primer) and ADJ014: 5′-TGTACGGTGGGAGGTCTAT-3′ (SEQ ID NO:7), (sense upstream vector primer)

The S99T variant was generated by classical two-step PCR, using ADJ013 and ADJ014 as vector primers, ADJ093 (5′-GTTCAGGTCTGTCACGGTGTAATTGGTCAG-CTT-3′) (SEQ ID NO:8) and ADJ094 (5′-AAGCTGACCAATTACACCGTGACAGA-CCTGAAC-3′) (SEQ ID NO:9) as mutation primers, and pIGY-22 as template. The 447 bp PCR product was subcloned into pcDNA3.1/Hygro (InVitrogen) using BamHI and XbaI, leading to plasmid pIGY-48.

pIGY-48 was transfected into CHO K1 cells by use of Lipofectaim2000 (Life Technologies) as transfection agent. 24 hours later the culture medium was harvested and assayed for interferon gamma activity. Using the Primary assay described herein, the following activity was obtained: 5.1×10⁶ AU/ml.

The E38N+S40T+S99T variant was generated by classical two-step PCR, using ADJ013 and ADJ014 as vector primers, ADJ091 (5′-CATGATCTTCCGATCGGTCTC-GTTCTTCCAATT-3′) (SEQ ID NO:10), and ADJ092 (5′-AATTGGAAGAACGAGACC-GATCGGAAGATCATG-3′) (SEQ ID NO:11), as mutation primers, and pIGY-48 as template. The 447 bp PCR product was subcloned into pcDNA3.1/Hygro (InVitrogen) using BamHI and XbaI, leading to plasmid pIGY-54.

pIGY-54 was transfected into CHO K1 cells by use of Lipofectaim2000 (Life Technologies) as transfection agent. 24 hours later the culture medium was harvested and assayed for interferon gamma activity. Using the Primary assay described herein, an activity of 1.3×10⁷ AU/ml was obtained.

Using similar standard techniques as described above, a number of full-length interferon gamma glycosylation variants were prepared.

Generation of C-Terminally Truncated Interferon Gamma Variants

C-terminally truncated interferon gamma variants, containing a stop codon immediately downstream of the codon for Leu135, were generated by one-step PCR using pIGY-22, pIGY-48 and pIGY-54 as templates, followed by subcloning of the PCR products into pcDNA3.1/Hygro (InVitrogen) using BamHI and XbaI. The primers used for construction of these variants were: ADJ014 (SEQ ID NO:7, upstream) and 5′-GAGTCTAGATTACAGCATCTGGCTTCTCTT-3′ (SEQ ID NO: 12, downstream). The resulting plasmids were termed pIGY-72 (wild-type interferon gamma truncated after Leu135), pIGY-73 (S99T variant truncated after Leu135) and pIGY-74 (E38N+S40T+S99T truncated after Leu135).

Generation of Cysteine-Containing Interferon Gamma Variants

Interferon gamma variants containing cysteine residues were generated using Stratagene's QuikChange™XL site-directed mutagenesis kit, according to the manufacturer's specifications. Seven interferon gamma variants, each containing one introduced cysteine, were generated using pIGY-48 as template: N10C+S99T, N16C+S99T, E38C+S99T, N59C+S99T, N83C+S99T, K94C+S99T and S99T+N104C. Similarly, six interferon gamma variants, each containing one introduced cysteine, were generated using pIGY-54 as template: N10C+E38N+S40T+S99T, N16C+E38N+S40T+S99T, E38N+S40T+N59C+S99T, E38N+S40T+N83C+S99T, E38N+S40T+K94C+S99T and E38N+S40T+S99T+N104C.

### Example E

### PEGylation of Cysteine-Containing Variants

All buffers were de-oxidized prior to use. Protein concentrations were estimated by measuring A280.

PEGylation Using the OPSS Coupling Chemistry

7.2 ml of 1.3 mg/ml of the interferon gamma variant N16C+S99T (full-length) in 5 mM sodium succinate, 4% mannitol, 0.01% Tween 20, pH 6.0, was reduced by incubation with 300 μl 0.5 M DTT for 30 minutes at room temperature. The interferon gamma variant was desalted by running 3 aliquots of 2.5 ml on a NAP25 gel filtration column (Pharmacia) in buffer A (50 mM sodium phosphate, 1 mM EDTA, pH 8.1). Each aliquote eluted in 3.5 ml.

mPEG-OPSS (10 KDa) was dissolved in buffer A to a concentration of 2 mg/ml and added in equal volume to the reduced and desalted interferon gamma variant and incubated for 60 min with gentle shaking at room temperature.

11 ml of the reaction mixture was concentrated to 1-6 ml using a Vivaspin20 column (VivaScience) and remaining mPEG was removed by gel filtration using a Sephacryl S-100 column (Pharmacia) equilibrated in buffer A.

The PEGylated interferon gamma variant was diafiltered into 5 mM sodium succinate, 4% mannitol, pH 6.0 using a Vivaspin 6 column (VivaScience) and Tween 20 was added to 0.01%. The purified PEGylated interferon gamma variant had a specific activity of 1.3×10⁶ AU/mg as measured in the Primary Assay described herein (15% of the specific activity of the corresponding non-PEGylated interferon gamma variant).

PEGylation Using the MAL Coupling Chemistry

1.6 ml of 1.5 mg/ml of the interferon gamma variant N59C+S99T (full-length) in 5 mM sodium succinate, 4% mannitol, 0.01% Tween 20, pH 6.0 was reduced by incubation with 64 μl 0.5 M DTT for 30 minutes at room temperature. The interferon gamma variant was desalted on a NAP25 gel filtration column (Pharmacia) in buffer A (50 mM sodium phosphate, 1 mM EDTA, pH 8.1). The interferon gamma variant eluted in 3.5 ml.

mPEG-MAL (5 kDa) was dissolved in buffer A to a concentration of 0.5 mg/ml and added in equal volume to reduced and desalted interferon gamma variant and incubated for 120 minutes with gentle shaking at room temperature.

Ammonium sulphate was added to a concentration of 0.9 M and the PEGylated interferon gamma variant was applied onto a 1 ml Resource™ phenyl column (Pharmacia) equilibrated in buffer B (20 mM sodium phosphate, 0.9 M ammonium sulphate, pH 6.6). The column was washed with 5 column volumes of buffer B before elution of the bound PEGylated interferon gamma variant in a linear gradient from 0-50% buffer C (20 mM sodium phosphate, pH 6.6) over 30 column volumes. The PEGylated interferon gamma variant eluted around 0.6 M ammonium sulphate.

Fractions containing PEGylated interferon gamma variant were pooled and diafiltered into 5 mM sodium succinate, 4% mannitol, pH 6.0 using a Vivaspin 6 column (VivaScience) and Tween 20 was added to 0.01%. The purified PEGylated interferon gamma variant had a specific activity of 2.4×10⁶ AU/mg as measured in the Primary Assay described herein (15% of the specific activity of the corresponding non-PEGylated interferon gamma variant).

### Example F

### Expression of Interferon Gamma Polypeptides in Mammalian Cells

For transient expression of interferon gamma, cells were grown to 95% confluency in media (Dulbecco's MEM/Nut.-mix F-12 (Ham) L-glutamine, 15 mM Hepes, pyridoxine-HCl (Life Technologies Cat # 31330-038)) containing 1:10 fetal bovine serum (BioWhittaker Cat # 02-701F) and 1:100 penicillin and streptomycin (BioWhittaker Cat # 17-602E). interferon gamma-encoding plasmids were transfected into the cells using Lipofectamine 2000 (Life Technologies) according to the manufacturer's specifications. 24 hrs after transfection, culture media were collected and assayed for interferon gamma activity. Furthermore, in order to quantify the relative number of glycosylation sites utilized, Western blotting was performed using harvested culture medium.

Stable clones expressing interferon gamma were generated by transfection of CHO K1 cells with interferon gamma-encoding plasmids followed by incubation of the cells in media containing 0.36 mg/ml hygromycin. Stably transfected cells were isolated and sub-cloned by limited dilution. Clones producing high levels of interferon gamma were identified by ELISA.

### Example G

### Large-Scale Production

Stable cell lines expressing interferon gamma or variants were grown in Dulbecco's MEM/Nut.-mix F-12 (Ham) L-glutamine, 15 mM Hepes, pyridoxine-HCl (Life Technologies Cat # 31330-038), 1:10 fetal bovine serum (BioWhittaker Cat # 02-701F), 1:100 penicillin and streptomycin (BioWhittaker Cat # 17-602E) in 1700 cm2 roller bottles (Corning, # 431200) until confluence. The media was then changed to 300 ml UltraCHO with L-glutamine (BioWhittaker Cat # 12-724Q) with the addition of 1:500 EX-CYTE VLE (Serological Proteins Inc. # 81-129) and 1:100 penicillin and streptomycin (BioWhittaker Cat # 17-602E). After 48 hours of growth, the media was replaced with fresh UltraCHO with the same additives. After another 48 hours of growth, the media was replaced with Dulbecco's MEM/Nut.-mix F-12 (Ham) L-glutamine, pyridoxine-HCl (Life Technologies Cat # 21041-025) with the addition of 1:100 ITS-A (Gibco/BRL # 51300-044), 1:500 EX-CYTE VLE (Serological Proteins Inc. # 81-129) and 1:100 penicillin and streptomycin (BioWhittaker Cat # 17-602E). Subsequently, every 24 h, culture media were harvested and replaced with 300 ml of fresh serum-free media with the same additives. The collected media were filtered through 0.22 μm filters to remove cells.

### Example H

### Purification

The filtrate was microfiltrated (0.22 μm) before ultrafiltration to approximately 1/15 volume using a Millipore TFF system. On the same system the concentrate was diafiltrated using 10 mM Tris, pH 7.6. Ammonium sulphate was added to a concentration of 1.7 M and after stirring the precipitate was removed by centrifugation at 8000 rpm for 25 minutes in a Sorvall centrifuge using a GS3 rotor.

The supernatant was applied onto a 25 ml Phenyl High Performance (Pharmacia) column previously equilibrated in 10 mM Tris, 1.7 M ammonium sulphate, pH 7.6. After application the column was washed with 3 column volumes of 10 mM Tris, 1.7 M ammonium sulphate, pH 7.6 and the bound interferon gamma variant was then eluted in a linear gradient over 10 column volumes to 100% 10 mM Tris, pH 7.6. The flow-through as well as the eluted interferon gamma variant was fractionated. Fractions enriched in the interferon gamma variant were pooled and buffer exchanged by diafiltration into 10 mM Tris, pH 9.0, using a Vivaflow200 system (VivaScience) with a molecular weight cut-off of 10,000 Da.

The interferon gamma variant was then applied onto a 18 ml Q-sepharose Fast Flow (Pharmacia) column previously equilibrated in 10 mM Tris, pH 9.0. After application the column was washed with 3 column volumes of 10 mM Tris, pH 9.0 before eluting the bound interferon gamma variant in a gradient from 0-100% 10 mM Tris, 0.5 M NaCl, pH 9.0, over 15 column volumes. The flow-through as well as the eluted interferon gamma variant was fractionated. Fractions enriched in the interferon gamma variant were pooled and buffer exchanged into 10 mM sodium phosphate, pH 7.0, by diafiltration using a Vivaspin20 (VivaScience) column with a molecular weight cut-off of 10,000 Da.

Then, the interferon gamma variant was applied onto an 8 ml CHT ceramic hydroxyapatite column (Biorad) previously equilibrated in 10 mM sodium phosphate, pH 7.0. After application the column was washed with 5 column volumes of 10 mM sodium phosphate, pH 7.0, before elution of the bound interferon gamma variant in a gradient from 0-60% 500 mM sodium phosphate, pH 7.0, over 30 column volumes. The flow-through as well as the eluted interferon gamma variant was fractionated. Fractions containing the interferon gamma variant were pooled and buffer exchanged into 5 mM sodium succinate, 4% mannitol, pH 6.0, using a VivaSpin20 column (VivaScience) and Tween 20 was subsequently added to a concentration of 0.01%. The interferon gamma variant was sterile filtered and stored at −80° C.

Alternatively, the interferon gamma variants may be purified according to the below purification scheme:

The filtrate is microfiltrated (0.22 μm) before ultrafiltration to approximately 1/15 volume using a Millipore TFF system. On the same system the concentrate is diafitrated using 10 mM Tris, pH 7.6, after which pH is adjusted to 9.0 and precipitate is removed by microfiltration.

The sample is applied onto a Q-sepharose Fast Flow (Pharmacia) column previously equilibrated in 10 mM Tris, pH 9.0. After application the column is washed with 3 column volumes of 10 mM Tris, pH 9.0 before eluting the bound interferon gamma variant in a gradient from 0-100% 10 mM Tris, 0.5 M NaCl, pH 9.0 over 15 column volumes. The flow-through as well as the eluted interferon gamma variant is fractionated. Fractions enriched in the interferon gamma variant are pooled, and pH is adjusted to 7.6. Ammonium sulphate is added to 1.5 M and after stirring the precipitate is removed by centrifugation.

The interferon gamma variant is then applied onto a Phenyl Sepharose High Performance (Pharmacia) previously equilibrated in 10 mM Tris, 1.5 M ammonium sulphate, pH 7.6.

After application the column is washed with 3 column volumes of 10 mM Tris, 1.5 M ammonium sulphate, pH 7.6, and the bound interferon gamma variant is then eluted in a linear gradient over 10 column volumes to 100% 10 mM Tris, pH 7.6. The flow-through as well as the eluted interferon gamma variant is fractionated. Fractions enriched in the interferon gamma variant are pooled and ammonium sulphate is adjusted to 1.7 M.

Then, the interferon gamma variant is applied onto a Butyl Sepharose column previously equilibrated in 10 mM sodium phosphate, 1.7 M ammonium sulphate, pH 7.6. After application the column is washed with 10 mM sodium phosphate, 1.7 M ammonium sulphate, pH 7.6, before eluting the bound interferon gamma variant in a step using 10 mM sodium phosphate, pH 6.5. The flow-through as well as the eluted interferon gamma variant is fractionated.

Fractions enriched in the interferon gamma variant are then pooled and applied onto a hydroxyapatite column previously equilibrated in 10 mM sodium phosphate, pH 6.5. After application the column is washed with 5 column volumes of 10 mM sodium phosphate, pH 6.5, before eluting the bound interferon gamma variant in a linear gradient from 0-100% 500 mM sodium phosphate, pH 6.5, over 30 column volumes. The flow-through as well as the eluted interferon gamma variant is fractionated.

Fractions containing the interferon gamma variant are pooled and buffer exchanged into a buffer containing 5 mM sodium succinate, 4% mannitol, pH 6.0. Tween 20 is subsequently added to a concentration of 0.01%. The interferon gamma variant is sterile filtered and stored at −80° C.

Formulation of Interferon Molecules

### Example 1

Formulation of Interferon Beta (IFN-Beta) Variant Q49N+Q51 T+F111N+R113T

The variant was constructed and expressed as described in Examples 7 and 8 of WO 01/15736 and purified with the following two-step procedure:

The harvested media from roller bottles was centrifuged and filtered through a 0.22 um filter (PVDF). The filtrated media was diafiltrated on a Vivaflow 200 system equipped with a polyethersulfon (PES) membrane with cut off 10000 and applied to a S-Sepharose column (Pharmacia) equilibrated with 50 mM sodium acetate, 50 mM sodium chloride, pH 5.5. The interferon variant bound to the column was eluted with 50 mM sodium acetate, 0.5 M sodium chloride, pH 5.5. The concentration of sodium chloride in the eluate from the S-Sepharose column was adjusted to 1.0 M and the sample was applied on a Phenyl-Sepharose High Performance column (Pharmacia) equilibrated with 50 mM sodium acetate, 1.0 M sodium chloride, pH 5.5. Following application the column was washed with Milli Q water. The interferon β variant was eluted with a gradient from Milli Q water to 60% ethylene glycol, 50 mM sodium acetate, pH 5.5 in 30 column volumes. Fractions containing fully glycosylated interferon β variant were collected. The buffer in the preparation was changed to 50 mM sodium phosphate, pH 7.0 in a Vivaspin 20 ml concentrator equipped with a PES membrane with cut off 10000.

The purified IFN-beta variant was formulated into a composition comprising the variant in an initial concentration of 10 MIU/ml within a 50 mM sodium phosphate buffer (finally adjusted to pH 7.0) and holding 35 mg/ml mannitol as well as 2 mg/ml Tween 80. One composition (Composition A) was without addition of Captisol® (available from CyDex Inc.). The other composition (Composition B) comprised 10 mg/ml of Captisol®

The compositions were stored in 0.5 ml Eppendorf tubes in aliquots of 50 μl (with no purging with either nitrogen or argon) at −80° C. and 35° C., respectively, for a period of 18 days. The antiviral activity was measured using the antiviral assay described in WO 01/15736.

The results are presented as “mean percent activity for samples stored at 35° C., as a function of activity of samples stored at −80° C. (analyzed at the same day)”

“% activity at 35° C. vs −80° C.”

Composition

| Days of storage | A | B |
|---|---|---|
| 10 | 24 | 144 |
| 18 | 27 | 78 |

### Example 2

Differential Scanning Calorimetry of Formulations Containing IFN-Beta Variant Q49N+Q51T+F111N+R113T

Samples of the protein of example 1 were analyzed by Differential Scanning Calorimetry (DSC) to study the unfolding (or denaturation) of the protein and especially determine the unfolding temperature (Tm) for the protein in each run.

The starting material for DSC analysis was a solution of the protein in 50 mM sodium acetate buffer (finally adjusted to pH 5.5). A series of solutions was prepared with varying excipients added to give a final protein concentration of 0.4 mg/mL. Water for Injection was used to serve as blanks for DSC, since the focus was only to determine shifts in Tm values. Prior to being subjected to DSC analysis, all solutions were degassed by using vacuum for a sufficient time period as described by MicroCal Inc.

The behavior of the protein was evaluated by using a DSC apparatus from MicroCal Inc (model VP-DSC). The temperature of the solution in question was gradually increased from ambient temperature (25° C.) to about 120° C., at a rate of 1.5° C. per minute. As the temperature increased two events occurred. The first event was an unfolding reaction (endothermic), and was observed as an upward peak in the scans. The second event was a precipitation (exothermic reaction), and was observed as a downward peak in the scans.

Adding either 2.4 mg/ml Tween 80; 5 mg/ml sodium chloride or 40 mg/ml Captisol® to the original solution resulted in a shift in the Tm-value of ΔTm: −0.7; +1.2 or +7.2° C., respectively. Here ΔTm is defined as:

ΔTm=(Tm₂−Tm₁)

wherein “Tm1” is related to the DSC scan of the original solution without additional excipients added and “Tm2” is related to each of the DSC scans of the solutions to which either 2.4 mg/ml Tween 80; 5 mg/ml sodium chloride or 40 mg/ml Captisol® is added.

These data clearly demonstrate that addition of Captisol® stabilize the protein being in solution; and thereby supporting the findings in Example 1.

### Example 3

Production, Purification and PEGylation of [C17S+Q49N+Q51T+D110F+F111N+R113T]IFN-Beta Glycosylation Variant.

A CHOK1 sub-clone (5/G-10) producing [C17S+Q49N+Q51T+D110F+F111N+R113T]IFN-beta glycosylation variant was seeded into 6 roller bottles, each with an expanded surface of 1700 cm² (Corning, USA), in 200 ml DMEM/F-12 medium (LifeTechnologies; Cat. # 31330) supplemented with 10% FBS and penicillin/streptomycin (P/S). After 2 days the medium was exchanged. After another 2 days the two roller bottles were nearly 100% confluent and the medium was shifted to 300 ml serum-free UltraCHO medium (BioWhittaker; Cat. # 12-724) supplemented with 1/500 EX-CYTE (Serologicals Proteins; Cat. # 81129N) and P/S. Growing the cells in this medium promotes a higher cell mass, higher than can be achieved in the serum containing medium. After 2 days the medium was renewed. After another 2 days the medium was shifted to the production medium: DMEM/F-12 medium (Life Technologies; Cat. # 21041) supplemented with 1/100 ITSA (Life Technologies; Cat. # 51300-044) [ITSA stands for Insulin (1.0 g/L)—Transferrin (0.55 g/L)—Selenium (0.67 mg/L) supplement for Adherent cultures], 1/500 EC-CYTE and P/S. The harvested media from the roller bottles were pooled before a medium sample was taken out for IFN-beta activity determination. Every day, in 21 days, 1.81 medium was harvested and frozen at −80° C.

The harvested media from roller bottles was centrifuged and filtered through a 0.22 μm filter (PVDF). The filtrated media was diafiltrated on a Vivaflow 200 system equipped with a polyethersulfon membrane with cut off 10000 and applied to a S-Sepharose column (Pharmacia).

The S-Sepharose column was equilibrated with 50 mM sodium acetate, 50 mM sodium chloride, pH 5.5 and the interferon variant was eluted with 50 mM sodium acetate, 0.5 M sodium chloride, pH 5.5. The concentration of sodium chloride in the eluate was adjusted to 1.0 M.

The eluate from the S-Sepharose column was applied on a Phenyl-Sepharose High Performance column (Pharmacia) equilibrated with 50 mM sodium acetate, 1.0 M sodium chloride, pH 5.5. Following application the column was washed with 50 mM sodium acetate, 50 mM sodium chloride, pH 5.5. The IFN-beta variant was eluted with a gradient from 50 mM sodium acetate, 50 mM sodium chloride, pH 5.5 to 60% ethylene glycol, 50 mM sodium acetate, pH 5.5 in 30 column volumes. Fractions containing fully glycosylated IFN-beta variant were collected and pooled.

The ethylene glycol in the eluate from the Phenyl-Sepharose was removed by passing the eluate through a S-Sepharose column equilibrated with 50 mM sodium acetate, 50 mM sodium chloride, pH 5.5. The ethylene glycol was in the flow through where as the interferon variant bound to the column. Following application the column was washed with 20 mM sodium acetate, pH 5.5 and the interferon variant was eluted with 100 mM sodium phosphate, pH 7.5.

The phosphate concentration in the eluate was adjusted to 15 mM sodium phosphate buffer, pH 7.2, and applied on a hydroxyapatite column (CHT I, Ceramic hydroxyapatite, Type I, Biorad) equilibrated with 15 mM sodium phosphate, pH 7.2. The fully glycosylated form passed through the column where as the underglycosylated form with one extra site used bound to the column and was eluted with a linear sodium phosphate gradient from 15 mM to 200 mM sodium phosphate, pH 6.8 in 20 column volumes. The purity of the fully glycosylated variant [C17S+Q49N+Q51T+D110F+F111N+R113T]IFN-beta was judged to be higher than 95% based on SDS-PAGE.

Following purification the variant was PEGylated. A fresh stock solution of 10 mg/ml SCM-PEG (succinimidyl ester of carboxymethylated PEG from Shearwater, Ala., 12 K or 20 K) was prepared in 96% ethanol before each experiment.

A protein solution of 0.1 mg/ml in 20 mM sodium phosphate, pH 7.0 was PEGylated with SCM-PEG, 20K, with 0.75 times molar surplus of PEG to possible PEGylation sites, i.e. lysines and N-terminus. After incubation for 30 min at room temperature, the reaction was quenched by addition of a surplus of 20 mM glycine, pH 8.0. The reaction mixture contained a mixture of mono-, di- and un-pegylated material. Mono-pegylated material was separated from other species using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing mono-pegylated material were pooled and characterized further.

In another experiment a protein solution of 0.16 mg/ml in 20 mM sodium phosphate, pH 7.0 was PEGylated with SCM-PEG, 12K, with 2 times molar surplus of PEG to possible PEGylation sites, i.e. lysines and N-terminus. After incubation for 30 min at room temperature, the reaction was quenched by addition of a surplus of 20 mM glycine, pH 8.0. The reaction mixture contained a mixture of mono-, di-, tri-pegylated material together with underivatized material. The pegylated material was separated from the unmodified protein using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing the mixture of mono-, di- and tri-pegylated protein were pooled and characterized further.

### Example 4

5 Differential Scanning Calorimetry of Formulations Containing [C17S+Q49N+Q51T+D110F+F111N+R113T]IFN-Beta Glycosylation Variant

Samples of the IFN-beta glycosylation variant (prepared under example 3) were analyzed by Differential Scanning Calorimetry (DSC) to study the unfolding (or denaturation) of the protein and especially determine the unfolding temperature (Tm) for the protein in each run.

The starting materials for DSC analysis were a solution of the protein in a 20 mM sodium phosphate (finally adjusted to pH 7.1). Solutions were prepared with varying excipients added to give a final protein concentration of 0.4 mg/mL. Water for Injection was used to serve as blanks for DSC, since the focus was only to determine shifts in Tm values. Prior to being subjected to DSC analysis, all solutions were degassed by using vacuum for a sufficient time period as described by MicroCal Inc.

The behavior of the protein was evaluated by using a DSC apparatus from MicroCal Inc (model VP-DSC). The temperature of the solution in question was gradually increased from ambient temperature (25° C.) to about 120° C., at a rate of 1.5° C. per minute. As the temperature increased an unfolding reaction (endothermic) was observed as an upward peak in the scans.

Comparing DSC runs of solution to which either “0.2 M mannitol” or “0.2 M mannitol+35 mg/ml Captisol®” revealed an increase in the Tm-value of about 6.4° C. for the sample containing Captisol®. These data clearly demonstrate that addition of Captisol® stabilize the protein being in solution.

### Example 5

Formulations Containing [C17S+Q49N+Q51T+D110F+F111N+R113T]IFN-Beta Glycosylation Variant PEGylated with 12 kDa.

The purified IFN-beta glycosylation variant PEGylated with 12 kDa (prepared under example 3 formulated into the following compositions comprising the variant in an initial concentration of about 5 MIU/ml within the following buffers: a 10 mM sodium acetate buffer (finally adjusted to pH 5.5) and holding 45 mg/ml mannitol as well as 2 mg/ml Tween buffer I), or a 50 mM sodium phosphate buffer (finally adjusted to pH 7.0) and holding 30 mg/ml mannitol as well as 2 mg/ml Tween 80 (buffer II). For buffer system I: one composition (Composition A) was without addition of Captisol®. The other composition (Composition B) comprised 10 mg/ml of Captisol®. For buffer system II: one composition (Composition C) was without addition of Captisol®. The other composition (Composition D) comprised 10 mg/ml of Captisol®.

The compositions were stored for a varying length of time period in 0.5 ml Eppendorf tubes in aliquots of 50 μl (with no purging with either nitrogen or argon) at −80° C. Samples of at least 0.4 mL were stored in siliconized glass vials (Type I glass) at 5, 25 and 35° C. (purged with nitrogen prior to closure).

The antiviral activity was measured using the antiviral assay described in WO 01/15736.

The results of the antiviral activity assay are shown in the table below as “mean percent activity for samples stored at the given temperature, as a function of activity of samples stored at −80° C. (analyzed at the same day)”:

| Storage temp.(° C.) | 5 | | | | 25 | | | | 35 | | | |
|---|---|---|---|---|---|---|---|---|---|---|---|---|
| Composition | A | B | C | D | A | B | C | D | A | B | C | D |
| Days of storage | | | | | | | | | | | | |
| 4 | — | — | — | — | — | — | — | — | 132 | 83 | 33 | 35 |
| 21 | — | — | — | — | — | — | — | — | 0 | 22 | 5 | 0 |
| 32 | — | — | — | — | — | — | — | — | 0 | 10 | 3 | 1 |
| 38 | 114 | 173 | 86 | 73 | 48 | 90 | 29 | 16 | — | — | — | — |
| 67 | 64 | 107 | — | — | 0 | 20 | — | — | — | — | — | — |
| 80 | 45 | 69 | — | — | — | 9 | — | — | — | — | — | — |

NB. If samples of the compositions are not analyzed at the given time points, it is indicated as: “—”.

These data clearly demonstrate that addition of Captisol® to a pharmaceutical acceptable buffer system can delay the loss of bioactivity at certain pH values.

In addition, visual inspection of the various compositions stored in vials have revealed that compositions containing Captisol® either prevents or delays the precipitation of the pegylated IFN-beta variant. For example, composition A and B stored for 34 days at 35° C. were inspected to be “highly turbid” and “a clear solution”, respectively.

### Example 6

Formulations Containing [C17S+Q49N+Q51T+D110F+F111N+R113T]IFN-Beta Glycosylation Variant PEGylated with 20 kDa.

The purified IFN-beta variant PEGylated with 20 kDa (prepared under example 3) was formulated into the following compositions comprising the variant in an initial concentration of 11-14 MIU/ml within the following buffers: a 10 mM sodium acetate buffer (finally adjusted to pH 5.5) and holding 45 mg/ml mannitol as well as both 2 mg/ml Tween 80 and 6.7 mg/ml Captisol® (Composition A), or a 50 mM sodium phosphate buffer (finally adjusted to pH 7.0) and holding 30 mg/ml mannitol as well as 2 mg/ml Tween 80 and 10 mg/ml Captisol® (Composition B).

The compositions were stored for a varying length of time period in 0.5 ml Eppendorf tubes in aliquots of 50 μl (with no purging with either nitrogen or argon) at −80° C. Samples of at least 0.4 mL were stored in siliconized glass vials (Type I glass) at 5, 25 and 35° C. (purged with nitrogen prior to closure).

The antiviral activity was measured using the antiviral assay described in WO 01/15736.

The results of the antiviral activity assay are shown in the table below as “mean percent activity for samples stored at the given temperature, as a function of activity of samples stored at −80° C. (analyzed at the same day)”:

| Storage | | | | | | |
|---|---|---|---|---|---|---|
| temp.(° C.) | 5 | | 25 | | 35 | |
| Composition | A | B | A | B | A | B |
| Days of storage | | | | | | |
| 4 | — | | — | — | 137 | 116 |
| 21 | — | — | — | — | 19 | 1 |
| 32 | — | — | — | — | 6 | 1 |
| 38 | 144 | 114 | 105 | 24 | — | — |
| 67 | 101 | 103 | 69 | — | — | — |
| 80 | 117 | 101 | 52 | 1 | — | — |
| 108 | 130 | 89 | — | — | — | — |

NB. If samples of the compositions are not analyzed at the given time points, it is indicated as: “—”.

These data clearly support the findings in Example 5 where it is shown that addition of Captisol® to a pharmaceutical acceptable buffer system can delay or even prevent the loss of bioactivity at certain pH values.

### Example 7

Production, Purification and PEGylation of [C17S+K19R+K33R+K45R+Q49N+F+F111N+R113T]IFN-Beta Glycosylation Variant.

CHOK1 sub-clone (5/G-10) producing [C17S+K19R+K33R+K45R+Q49N+Q51T+D110F+F1111N+R113T]IFN-beta glycosylation variant was produced in 6 roller bottles as described in example 3 and purified according to the protocol used in example 3. The purity of the fully glycosylated variant [C17S+K19R+K33R+K45R+Q49N+Q51T+D110+F111N+R113T]IFN-beta was judged to be higher than 95% based on SDS-PAGE.

Following purification the variant was PEGylated. A fresh stock solution of SCM-PEG (succinimidyl ester of carboxymethylated PEG from Shearwater, Ala., 12 kD or 20 kD) was prepared in ethanol before each experiment.

A protein solution of 0.1 mg/ml in 20 mM sodium phosphate, pH 7.0 was PEGylated with SCM-PEG, 20K, with 3 times molar surplus of PEG to possible PEGylation sites, i.e. lysines and N-terminus. After incubation for 30 min at room temperature, the reaction was quenched by addition of a surplus of 20 mM glycine, pH 8.0. The reaction mixture contained mixture of mono-, di- and un-pegylated material. Mono-pegylated material was separated from other species using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S— 100, ((16/60) Pharmacia) equilibrated with 100 mM sodium acetate, 200 mM sodium chloride, pH 5.5. Fractions containing mono-pegylated material was pooled and characterized further.

In another experiment a protein solution of 0.1 mg/ml in 20 mM sodium phosphate, pH 7.0 was PEGylated with (10 mg/ml) SCM-PEG, 12K, with 5 times molar surplus of PEG to possible PEGylation sites, i.e. lysines and N-terminus. After incubation for 30 min at room temperature, the reaction was quenched by addition of a surplus of 20 mM glycine, pH 8.0. The reaction mixture contained a mixture of mono-, di-, tri-pegylated material together with underivatized material. The pegylated material was separated from the unmodified protein using either cation exchange chromatography or size-exclusion chromatography or a combination of both. pH in the PEGylation solution was adjusted to pH 2.7 and the sample was applied on a SP-Sepharose HR (Pharmacia) column equilibrated with 20 mM sodium citrate, pH 2.7. The pegylated protein was eluted from the column with 50 mM sodium acetate containing 1 M sodium chloride and applied on a size-exclusion column, Sephacryl S-100, ((16/60) Pharmacia) equilibrated with 100 mM sodium acetate, 200 mM sodium chloride, pH 5.5. Fractions containing the mixture of mono-, di- and tri-pegylated protein were pooled and characterized further.

### Example 8

Formulations Containing [C17S+K19R+K33R+K45R+Q49N+Q51T+D110F+F111N+R113T]IFN-Beta Glycosylation Variant PEGylated with 20 kDa.

The purified IFN-beta variant PEGylated with 20 kDa (prepared under example 7) was formulated into the following compositions comprising the variant in an initial concentration of 5-10 MIU/ml within the following buffers: a 10 mM sodium acetate buffer (finally adjusted to pH 5.0, “buffer A”): a 10 mM sodium acetate buffer (finally adjusted to pH 5.5, “buffer B”), a 10 mM sodium succinate buffer (finally adjusted to pH 5.5, “buffer C”), a 10 mM sodium succinate buffer (finally adjusted to pH 6.0, “buffer D”) and a 10 mM sodium citrate buffer (finally adjusted to pH 6.0, “buffer E”). Various combinations of the following three excipients Tween 80 (none, 0.2 and 2.0 mg/ml), Captisol® (10 and 50 mg/ml) and mannitol (17 and 39 mg/ml) were added for each of the five mentioned buffer systems. In addition, for buffer system C and E combinations without Tween 80 or Captisol® but only mannitol added (34 and 32 mg/ml, respectively) were also investigated.

The amount of mannitol was adjusted to ensure isotonic solutions suitable for parenteral administration.

The compositions were stored for a varying length of time period in 0.5 ml Eppendorf tubes in aliquots of 25 μl (with no purging with either nitrogen or argon) at −80° C. and 5° C. Samples of at least 0.4 ml were stored in siliconized glass vials (Type I glass) at 25 (purged with nitrogen prior to closure).

The antiviral activity was measured using the antiviral assay described in WO 01/15736.

Results of the antiviral activity assay are shown in the following tables as “mean percent activity for samples stored at the given temperature, as a function of activity of samples stored at −80° C. (analyzed at the same day)”.

Seven different formulations all containing 10 mM sodium succinate buffer and mannitol (finally adjusted to pH 5.5, “buffer C”) where stored at −80 and 25° C. before antiviral analysis. The formulation without Tween 80 and Captisol® was put on stability 20 days later than the other formulations.

| Captisol | Tween 80 (mg/ml) | | |
|---|---|---|---|
| (mg/ml) | 0 | 0.2 | 2 |
| 0 | 40* | | |
| 10 | 64 | 101 | 116 |
| 50 | 117 | 109 | 65 |

Mean percent activity for samples of buffer C compositions stored for 94-95 days at 25° C., as a function of activity of samples stored at −80° C. (analyzed at the same day).
*Mean percent activity for samples of this buffer C composition stored for 74 days at 25° C., as a function of activity of samples stored at −80° C. (analyzed at the same day).

| Captisol | Tween 80 (mg/ml) | | |
|---|---|---|---|
| (mg/ml) | 0 | 0.2 | 2 |
| 0 | 17* | | |
| 10 | 120 | 72 | 41 |
| 50 | 96 | 91 | 116 |

Mean percent activity for samples of buffer C compositions stored for 160 days at 25° C., as a function of activity of samples stored at −80° C. (analyzed at the same day).
*Mean percent activity for samples of this buffer C composition stored for 140 days at 25° C., as a function of activity of samples stored at −80° C. (analyzed at the same day).

Seven different formulations all containing 10 mM sodium citrate buffer and mannitol (finally adjusted to pH 6.0, “buffer E”) where stored at −80 and 25° C. before without Tween 80 and Captisol® was put on stability 20 days later than the other formulations.

| Captisol | Tween 80 (mg/ml) | | |
|---|---|---|---|
| (mg/ml) | 0 | 0.2 | 2 |
| 0 | 68* | | |
| 10 | 86 | 81 | 61 |
| 50 | 103 | 117 | 111 |

Mean percent activity for samples of buffer E compositions stored for 94-95 days at 25° C., as a function of activity of samples stored at −80° C. (analyzed at the same day).
*Mean percent activity for samples of this buffer E composition stored for 75 days at 25° C., as a function of activity of samples stored at −80° C. (analyzed at the same day).

| Captisol | Tween 80 (mg/ml) | | |
|---|---|---|---|
| (mg/ml) | 0 | 0.2 | 2 |
| 0 | 25* | | |
| 10 | 83 | 45 | 8 |
| 50 | 122 | 80 | 45 |

Mean percent activity for samples of buffer E compositions stored for 164 days at 25° C., as a function of activity of samples stored at −80° C. (analyzed at the same day).
*Mean percent activity for samples of this buffer E composition stored for 144 days at 25° C., as a function of activity of samples stored at −80° C. (analyzed at the same day).

Considering that the compositions that do not contain Captisol® have been stored for a considerably shorter time period than the remaining compositions, the data clearly show that addition of Captisol® to a pharmaceutical acceptable buffer system can delay or even prevent the loss of bioactivity at certain pH values—even when stored at elevated temperatures for an extended time period.

### Example 9

Stability of Selected Purified IFN-Beta Variants

The stability of selected purified IFN-beta variants, including the specific variants of examples 1-8, are investigated within combinations of the following parameters:

a) “Variant concentration” selected from 1-50 MIU/ml.

b) Buffer type selected from sodium acetate, sodium succinate, sodium citrate, sodium maleate, sodium carbonate, sodium tartrate, sodium lactate, and mixtures thereof, having a suitable concentration up to 100 mM for each buffer type.

c) pH range selected from pH 4.0-7.0.

d) Concentration of Captisol® selected from 5-100 mg/ml.

e) Type and amount of other relevant excipients selected from Tween 20 (up to 2 mg/ml), Tween 80 (up to 2 mg/ml), mannitol (up to 50 mg/ml), sodium chloride (up to 9 mg/ml).

f) Primary containers suitable for products given by parenteral administration.

### Example 10

Formulations Containing [C17S+Q49N+Q51 T+D110F+F111N+R113T]IFN-Beta Glycosylation Variant PEGylated with 20 kDa.

Prior to formulation the purified IFN-beta variant PEGylated with 20 kDa (prepared under example 3) was equilibrated with a solution consisting of 10 mM sodium acetate buffer (finally adjusted to pH 5.5) holding 25 mg/ml mannitol. This material was formulated into the following compositions comprising the variant in an initial concentration of 100 g/ml within the following buffers: a 10 mM sodium acetate buffer (finally adjusted to pH 5.5) holding mannitol, sodium chloride (NaCl), Tween 80 and Captisol® in the following combinations.

| | Tween 80 | Captisol ® | Mannitol | NaCl |
|---|---|---|---|---|
| Formulation | (mg/ml) | (mg/ml) | (mg/ml) | (mg/ml) |
| M01 | 0 | 0 | 48 | 0 |
| M02 | 0.05 | 0 | 48 | 0 |
| M03 | 0.20 | 0 | 48 | 0 |
| M04 | 0.05 | 10 | 45 | 0 |
| M05 | 0.20 | 10 | 45 | 0 |
| M06 | 0 | 25 | 38 | 0 |
| M07 | 0.05 | 25 | 38 | 0 |
| M08 | 0.20 | 25 | 38 | 0 |
| M09 | 0 | 50 | 28 | 0 |
| M10 | 0.05 | 50 | 28 | 0 |
| M11 | 0 | 100 | 9.3 | 0 |
| M12 | 0.05 | 100 | 9.3 | 0 |
| NM01 | 0 | 0 | 9.3 | 6.8 |
| NM02 | 0.05 | 0 | 9.3 | 6.8 |
| NM03 | 0.20 | 0 | 9.3 | 6.8 |
| NM04 | 0.05 | 10 | 9.3 | 6.2 |
| NM05 | 0.20 | 10 | 9.3 | 6.2 |
| NM06 | 0 | 25 | 9.3 | 5.1 |
| NM07 | 0.05 | 25 | 9.3 | 5.1 |
| NM08 | 0.20 | 25 | 9.3 | 5.1 |
| NM09 | 0 | 50 | 9.3 | 3.4 |
| NM10 | 0.05 | 50 | 9.3 | 3.4 |

The compositions were sterilized by filtration, filled under aseptic conditions into sterilized containers and stored for a varying length of time period. Aliquots of 20μl were filled into 0.5 ml Eppendorf tubes and stored at −80° C. Aliquots of at least 0.3 ml were filled into siliconized glass vials (Type I glass) and stored at 5, 25 and 35° C.

The antiviral activity was measured using the antiviral assay described in WO 01/15736.

The results of the antiviral activity assay after about 1 month storage are shown in the table below as “mean percent activity for samples stored at the given temperature, as a function of activity of samples stored at −80° C. (analyzed at the same day)”:

| Captisol | Tween 80 (mg/ml) | | |
|---|---|---|---|
| (mg/ml) | 0 | 0.05 | 0.2 |
| 0 | 76 | 65 | 56 |
| 10 | | 123 | 110 |
| 25 | 124 | 111 | 113 |
| 50 | 101 | 109 | |
| 100 | 155 | 128 | |

Mean percent activity for samples of formulation compositions M01 to M012 stored at 25° C., as a function of activity of samples stored at −80° C. (analyzed at the same day).

| Captisol | Tween 80 (mg/ml) | | |
|---|---|---|---|
| (mg/ml) | 0 | 0.05 | 0.2 |
| 0 | 38 | 16 | 7 |
| 10 | 71 | 68 | |
| 25 | 109 | 90 | 73 |
| 50 | 87 | 93 | |
| 100 | 115 | 100 | |

Mean percent activity for samples of formulation compositions M01 to M012 stored at 35° C., as a function of activity of samples stored at −80° C. (analyzed at the same day).

These data clearly support the findings in Example 5 where it is shown that addition of Captisol® to a pharmaceutical acceptable buffer system can delay or even prevent the loss of bioactivity at certain pH values.

Compared to Example 6, significantly lot less Tween 80 is used in this example, which may explain the observed improved stability at elevated temperatures of the molecule in question.

### Example 11

Formulations Containing Wild-Type IFN-Beta (IFN-Beta)

The IFN-beta bulk preparation was purified as the variant mentioned in Example 3 with the differences that the final step consisted of a gel filtration through a Superdex 75 column. This material was then equilibrated with a solution consisting of 50 mM sodium acetate (adjusted to pH 5.5) holding both 0.1 M sodium chloride and 0.2 M mannitol.

This material formulated into the following compositions comprising the IFN-beta in an initial concentration of about 5 MIU/ml within the following buffers: a 50 mM sodium acetate buffer (finally adjusted to pH 5.5) holding 28 mg/ml mannitol, 1.3 mg/ml sodium chloride, and 2 mg/ml Tween 80 as well as no Captisol® (Formulation A) or 10 mg/ml Captisol® (Formulation B).

The compositions were filled into Eppendorf tubes and stored for a varying length of time period in aliquots of 50 μl at −80° C., −20 and 5° C.

The antiviral activity was measured using the antiviral assay described in WO 01/15736.

The results of the antiviral activity assay after 354 days storage are shown in the table below as “mean percent activity for samples stored at the given temperature, as a function of activity of samples stored at −80° C. (analyzed at the same day)”:

| Storage | Formulation | Formulation |
|---|---|---|
| temperature | A | B |
| −20° C. | 32 | 35 |
| 5° C. | 9 | 31 |

Mean percent activity for samples of formulation compositions A and B stored for 354 days at −20° C. and 5° C., as a function of activity of samples stored at −80° C. (analyzed at the same day).

### Example 12

Formulations Containing Wild-Type IFN-Gamma (IFN-Gamma)

The IFN-gamma bulk preparation was conducted as described in Example H within the section “Materials and methods for preparing interferon gamma”. This preparation was formulated into the following compositions comprising the IFN-gamma in an initial concentration of 0.5 mg/ml within the following buffers: a 5 mM sodium succinate buffer (finally adjusted to pH 6.0) holding 40 mg/ml mannitol and 0.01% Tween 20 as well as no Captisol® (Formulation A) or 50 mg/ml Captisol® (Formulation B).

The compositions were sterilized by filtration, filled under aseptic conditions into sterilized containers and stored for a varying length of time period. Aliquots of 20 μl were filled into 0.5 ml Eppendorf tubes and stored at −80° C. Aliquots of at least 0.15 ml were filled into siliconized glass vials (Type I glass) and stored at 5, 25, 35 and 40° C.

The activity was measured using the luciferase assay described in the section “Materials and methods for preparing interferon gamma”.

The results of the luciferase assay after 8 days of storage are shown in the table below as “mean percent activity for samples stored at the given temperature, as a function of activity of samples stored at −80° C. (analyzed at the same day)”:

| Storage | Formulation | Formulation |
|---|---|---|
| temperature | A | B |
| 25° C. | 63 | 118 |
| 35° C. | 44 | 95 |
| 40° C. | 39 | 83 |

Mean percent activity for samples of formulation compositions A and B stored for 8 days at 25, 35 and 40° C., as a function of activity of samples stored at −80° C. (analyzed at the same day).

### Example 13

Formulations Containing [E38N+S40T+S99T]IFN-Gamma Glycosylation Variant

The IFN-gamma variant was prepared as described in Example D described in the section “Materials and methods for preparing interferon gamma”. This material was purified as described in Example H described in the section “Materials and methods for preparing interferon gamma”. The purified material formulated into the following compositions comprising the variant in an initial concentration of 0.5 mg/ml within the following buffers: a 5 mM sodium succinate buffer (finally adjusted to pH 6.0) holding 40 mg/ml mannitol and 0.01% Tween 20;as well as either no Captisol® (Formulation A) or 50 mg/ml Captisol® (Formulation B).

The compositions were sterilized by filtration, filled under aseptic conditions into sterilized containers and stored for a varying length of time period. Aliquots of 20 μl were filled into 0.5 ml Eppendorf tubes and stored at −80° C. Aliquots of at least 0.15 ml were filled into siliconized glass vials (Type I glass) and stored at 5 and 25.

The activity was measured using the luciferase assay described in the section “Materials and methods for preparing interferon gamma”.

The results of the luciferase activity assay are shown in the table below as “mean percent activity for samples stored at the given temperature, as a function of activity of samples stored at −80° C. (analyzed at the same day)”:

| | |
|---|---|
| Formulation A | Formulation B |

| Days of | Storage at | Storage at | Storage at | Storage at |
|---|---|---|---|---|
| storage | 5° C. | 25° C. | 5° C. | 25° C. |
| 6 | | 5 | | 35 |
| 14 | 47 | 0 | 72 | 9 |
| 28 | 13 | | 44 | |

Mean percent activity for samples of formulation compositions A and B stored at 5 and 25° C., as a function of activity of samples stored at −80° C. (analyzed at the same day).

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. For example, all the techniques and apparatus described above may be used in various combinations. All publications, patents, patent applications, and/or other documents cited in this application are incorporated herein by reference in their entirety for all purposes to the same extent as if each individual publication, patent, patent application, and/or other document were individually indicated to be incorporated herein by reference in its entirety for all purposes.

1. A stabilized composition comprising an interferon molecule and a sulfoalkyl ether cyclodextrin derivative.

2. The composition of claim 1, wherein the interferon molecule exhibits aggregate formation or loss of bioactivity during storage.

3. The composition of claim 1, wherein the interferon molecule exhibits aggregate formation and loss of bioactivity during storage.

4. The composition of claim 1, wherein the interferon molecule is a human wildtype interferon or a variant thereof.

5. The composition of claim 4, wherein the human wildtype interferon is selected from the group consisting of interferon alpha, interferon beta, interferon omega, interferon tau, interferon epsilon, and interferon gamma.

6. The composition of claim 4, wherein the interferon molecule is a variant of a human wildtype interferon selected from the group consisting of interferon alpha, interferon beta, interferon omega, interferon tau, interferon epsilon, and interferon gamma.

7. The composition of claim 1, wherein the interferon molecule further comprises at least one non-polypeptide moiety.

8. The composition of claim 7, wherein the interferon molecule is glycosylated.

9. The composition of claim 7, wherein the non-polypeptide moiety is a polymer molecule.

10. The composition of claim 9, wherein the polymer molecule is a polyethylene glycol (PEG) molecule.

11. The composition of claim 9, wherein the interferon molecule comprises at least one PEG molecule.

12. The composition of claim 7, wherein the interferon molecule is glycosylated and comprises at least one PEG molecule.

13. The composition of claim 7, wherein the interferon molecule is an interferon beta polypeptide comprising one PEG molecule.

14. The composition of claim 4, wherein the interferon molecule is an interferon beta polypeptide.

15. The composition of claim 14, wherein the interferon beta polypeptide is wildtype human interferon beta or a variant thereof.

16. The composition of claim 7, wherein the interferon molecule is a conjugate comprising an interferon beta polypeptide, the amino acid sequence of which differs from that of wildtype human interferon beta in at least one introduced glycosylation site, the conjugate further comprising at least one sugar moiety attached to the introduced glycosylation site.

17. The composition of claim 16 wherein the at least one introduced glycosylation site is an N-glycosylation site at a position selected from the group consisting of S2N+N4S/T, L6S/T, L5N+G7S/T, F8N+Q10S/T, L9N+R11S/T, R11N, R11N+S13T, S12N+N14S/T, F15N+C17S/T, Q16N+Q18S/T, Q18N+L20S/T, K19N+L21S/T, W22N+L24S/T, Q23N+H25S/T, G26N+L28S/T, R27N+E29S/T, L28S+Y30S/T, Y30N+L32S/T, L32N+D34S/T, K33N+R35S/T, R35N+N37S/T, M36N+F38S/T, D39S/T, D39N+P41S/T, E42N+I44S/T, Q43N+K45S/T, K45N+L47S/T, Q46N+Q48S/T, L47N+Q49T/S, Q48N+F50S/T, Q49N+Q51S/T, Q51N+E53S/T, K52N+D54S/T, L57N+159S/T, Q64N+I66S/T, A68N+F70S/T, R71N+D73S/T, Q72N, Q72N+S74T, D73N, D73N+S75T, S75N+T77S, S75N, S76N+G78S/T, E81N+I83S/T, T82N+V84S/T, E85N+L87S/T, L88S/T, A89N+V91S/T, Y92S/T, Y92N+Q94S/T, H₉₃N+195S/T, L98S/T, H₉₇N+K99S/T, K99N+V101 S/T, T100N+L102S/T, E 103N+K105S/T, E 104N+L106S/T, K105N+E107S/T, E107N+E109S/T, K108N+D110S/T, E109N+F111S/T, D110N+T112S, D110N, F111N+R113S/T, R113N+K115S/T, G114N+L116S/T, K 115N+M117S/T, L116N, L116N+S118T, S119N+H212S/T, L120N+L122S/T, H121N+K123S/T, K123N+Y125S/T, R124N+Y126S/T, G127N+I129S/T, R128N+L130S/T, L130N+Y132S/T, H131N+L133S/T, K134N+K136S/T, A135N+E137S/T, K136N+Y138S/T, E137N, Y138N+H140S/T, H140N+A142S/T, V148N+1150S/T, R152N+F154S/T, Y155N+1157S/T, L160S/T, R159N+T161S, R159N, G162N+L164S/T, and Y163N+R165S/T, the substitutions being indicated relative to the amino acid sequence of wildtype human interferon beta shown in SEQ ID NO:1.

18. The composition of claim 14, wherein the interferon beta polypeptide is an variant of wildtype human interferon beta comprising a C17S mutation, the substitution being indicated relative to the amino acid sequence of wildtype human interferon beta shown in SEQ ID NO:1.

19. The composition of claim 14, wherein the interferon molecule is an interferon beta polypeptide comprising a mutation selected from the group consisting of:
Q49N+Q51T;
F111N+R113T;
Q49N+Q51T+F111N+R113T;
C17S+Q49N+Q51T+L98P+F111N+R113T;
S2N+N4T+C17S+Q51N+E53T;
S2N+N4T+C17S+Q51N+E53T+F111N+R113T;
C17S+Q49N+Q51T+F111N+R113T;
C17S+Q49N+Q51T+D110F+F111N+R113T;
C17S+Q48F+Q49N+Q51T+D110F+F111N+R113T;
C17S+Q48Y+Q49N+Q51T+D110Y+F111N+R113T;
K19R+K45R+K123R;
K19R+K45R+Q49N+Q51T+F111N+R113T+K123R;
C17S+K19R+K45R+Q49N+Q51T+F111N+R113T+K123R;
C17S+K19R+K45R+Q49N+Q51T+F111N+R113T+K123R;
C17S+K19R+Q49N+Q51T+F111N+R113T+K123R;
C17S+K19R+K45R+Q49N+Q51T+D110F+F111N+R113T+K123R;
C17S+K19R+Q49N+Q51T+D110F+F111N+R113T+K123R;
S2N+N4T+C17S+K19R+K45R+Q51N+E53T+K123R;
C17S+K19R+K45R+Q48F+Q49N+Q51T+D110F+F111N+R113T+K123R;
S2N+N4T+C17S+K19R+K45R+Q51N+E53T+D110F+F110N+R113T+K123R;
C17S+K19R+K33R+K45R+Q49N+Q51T+D110F+F111N+R113T;
C17S+K19R+K33R+K45R+Q49N+Q51T+D110F+F111N+R113T+K123R;
C17S+K19R+K33R+K45R+Q49N+Q51T+F111N+R113T; and
C17S+K19R+K33R+K45R+Q49N+Q51T+F111N+R113T+K123R; the substitutions being indicated relative to the amino acid sequence of wildtype human interferon beta shown in SEQ ID NO:1.

20. The composition of claim 1, wherein the interferon molecule is an interferon gamma polypeptide.

21. The composition of claim 20, wherein the interferon gamma polypeptide is wildtype human interferon gamma or a variant thereof.

22. The composition of claim 21, wherein the interferon gamma polypeptide is a variant of human wildtype interferon gamma comprising the substitution S99T, the substitution being indicated relative to the amino acid sequence of wildtype human interferon gamma shown in SEQ ID NO:2.

23. The composition of claim 22, wherein the variant further comprises the substitutions E38N+S40T, the substitutions being indicated relative to the amino acid sequence of wildtype human interferon gamma shown in SEQ ID NO:2.

24. The composition of claim 20, wherein the interferon gamma polypeptide is C-terminally truncated by 1-15 amino acid residues.

25. The composition of claim 1, wherein the sulfoalkyl ether cyclodextrin derivative is a compound of the Formula (I): 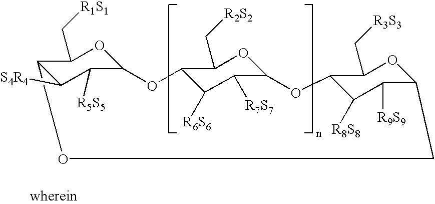
n is 4, 5 or 6,
R₁, R₂, R₃, R4, R₅, R6, R₇, R₈, and R9 are each, independently, —O— or a —O—(C₂-C₆ alkylene)-SO₃— group, wherein at least one of R₁ and R₂ is independently a —O—(C₂-C₆ alkylene)-SO₃-group, and
S₁, S₂, S₃, S₄, S₅, S₆, S₇, S8, and S₉ are each, independently, a pharmaceutically acceptable cation.

26. The composition of claim 25, wherein the sulfoalkyl ether cyclodextrin derivative is a beta cyclodextrin sulfobutyl ether or a salt form thereof.

27. The composition of claim 26, wherein the sulfoalkyl ether cyclodextrin derivative is a sodium salt of a beta cyclodextrin sulfobutyl ether.

28. The composition of claim 27, wherein the sulfoalkyl ether cyclodextrin derivative is Captisol®.

29. The composition of claim 1, wherein the sulfoalkyl ether cyclodextrin derivative is present in a concentration from 1 mg/ml to 150 mg/ml.

30. The composition of claim 1, wherein the interferon molecule is present in an amount corresponding to 1-100 MIU/ml of a liquid formulation or 1-100 MIU/dose of a solid formulation.

31. The composition of claim 30, wherein the interferon molecule is present in an amount corresponding to 1-50 MIU/ml of a liquid formulation.

32. The composition of claim 30, wherein the interferon molecule is present in an amount corresponding to 1-50 MIU/dose of a solid formulation.

33. The composition of claim 1, wherein the composition has a pH in the range of 4-8.

34. The composition of claim 33, wherein the composition has a pH in the range of 5-8.

35. The composition of claim 33, wherein the composition has a pH in the range of 4-7.

36. The composition of claim 1, further comprising a buffering agent.

37. The composition of claim 36 wherein the buffering agent is present in a concentration of up to 100 mM.

38. The composition of claim 1, wherein the composition is a liquid isotonic solution having an osmolarity of about 240-360 mOsmol/kg.

39. The composition of claim 1, further comprising a tonicity agent.

40. The composition of claim 1 which is in the form of a dry formulation, a liquid formulation, an aqueous solution, or an aqueous suspension.

41. The composition of claim 40, which is in the form of a frozen liquid formulation, a spray dried formulation, or a freeze-dried formulation.

42. The composition of claim 1, which is suitable for parenteral, nasal or pulmonary administration.

43. The composition of claim 42, which is suitable for intraveneous, intramusculary or subcutaneous administration.

44. The composition of claim 1, further comprising an excipient.

45. The composition of claim 1, further comprising a second stabilizing agent capable of reducing aggregation or chemical degradation of the interferon molecule.

46. The composition of claim 1, further comprising a preservating agent, a viscocity increasing agent, or a preservating agent plus a viscocity increasing agent.

47. The composition of claim 1, which lacks a preservating agent.

48. The composition of claim 1, further comprising HSA.

49. The composition of claim 1, which lacks HSA.

50. The composition of claim 1, further comprising a surfactant.

51. The composition of claim 50, wherein the surfactant is a non-ionic surfactant.

52. The composition of claim 1, which lacks a surfactant.

53. The composition of claim 1, wherein the interferon molecule has essentially retained its antiviral activity during
a) storage at a temperature of 37° C. for a period of at least 1 week, or
b) storage at a temperature of 25° C. for a period of at least 4 weeks.

54. A primary product container comprising the composition of claim 1.

55. The container of claim 54, which is a prefilled syringe.

56. A method for increasing stability of an interferon molecule formulated into a pharmaceutical composition, said method comprising incorporating into the composition a sulfoalkyl ether cyclodextrin derivative and optionally a buffering agent.

57. The method of claim 56, wherein the interferon molecule exhibits aggregate formation during storage and the sulfoalkyl ether cyclodextrin derivative is incorporated in an amount sufficient to reduce the aggregate formation of the interferon molecule.

58. A method of treating a mammal with a disease, disorder, or condition for which interferon is a useful treatment, comprising administering to the mammal an effective amount of the composition of claim 1.

59. The method of claim 58, wherein the interferon molecule is an interferon beta polypeptide, and the disease, disorder, or condition is selected from the group consisting of multiple sclerosis, hepatitis B, hepatitis C, Crohn's disease, ulcerative colitis, and a cancer.

60. The method of claim 58, wherein the interferon molecule is an interferon gamma polypeptide, and the disease, disorder, or condition is selected from the group consisting of an interstitial pulmonary disease, a granulomatous disease, a mycobacterial infection, kidney cancer, osteopetrosis, scleroderma, hepatitis B, hepatitis C, septic shock, and rheumatoid arthritis.
